(19) United States
(12) Patent Application Publication
(10) Pub. No.: US 2012/0183547 A1
(43) Pub. Date: Jul. 19, 2012

(54) COMPOSITIONS AND METHODS COMPRISING VEGFR-2 AND VEGFR-3 ANTAGONISTS FOR THE TREATMENT OF METASTATIC DISEASE

(75) Inventors: Mihaela Skobe, New York, NY (US); Suvendu Das, Bronx, NY (US)

(73) Assignee: MOUNT SINAI SCHOOL OF MEDICINE, New York, NY (US)

(21) Appl. No.: 13/322,447

(22) PCT Filed: May 27, 2010
(86) PCT No.: PCT/US10/36481

§ 371 Date: Mar. 29, 2012

Related U.S. Application Data
(60) Provisional application No. 61/217,188, filed on May 27, 2009.

(51) Int. Cl. A61K 39/395; A61P 35/00; A61N 5/00; A61K 38/17

(52) U.S. Cl. 424/135.1; 424/130.1; 424/133.1; 514/19.8; 600/1

(57) ABSTRACT

The invention is directed to methods for inhibiting growth of tumor metastases in lymph nodes, lungs and other distant organ sites comprising administering one or more VEGFR-3 antagonist(s) and optionally one or more VEGFR-2 antagonist(s).

This application claims priority from U.S. Provisional Application Ser. No. 61/217,188, filed on May 27, 2009, which is incorporated herein by reference in its entirety.

### GOVERNMENT SPONSORED RESEARCH OR DEVELOPMENT

This invention was made with government support under grant W81XWH-05-1-0392 awarded by DOD, BCRP. The government thus has certain rights in the invention.

### TECHNICAL FIELD OF THE INVENTION

The present invention is directed, generally, to the treatment of cancer. More specifically, disclosed herein are methods for inhibiting tumor metastases in lymph nodes, lungs, liver, kidneys, skin, peritoneum and other distant organ sites comprising administering one or more VEGFR-3 antagonist(s) and optionally one or more VEGFR-2 antagonist(s).

### BACKGROUND OF THE INVENTION

The metastatic spread of tumor cells is a major cause of death in cancer patients. The lymphatic system is the primary pathway of cancer metastasis and the spread of cancer cells via lymphatic vessels to the regional lymph nodes is one of the most important indicators of tumor aggressiveness. While research efforts over the past decades have primarily focused on understanding the mechanisms of angiogenesis and its significance for tumor growth and progression, the lymphatic vascular system has received little attention. Consequently, although the extent of lymph node involvement is a major determinant for the staging and prognosis of many types of cancer, the mechanisms by which cancer spreads via the lymphatic system remain poorly understood (Pepper et al., Cell Tissue Res 314:167-77 (2003); Alitalo and Carmeliet., Cancer Cell 1:219-27 (2002)).

The lymphatic system is comprised of capillaries and larger collecting vessels continuously lined by endothelial cells which return extravasated fluid and macromolecules from the interstitial space back to the blood circulation (Alitalo and Carmeliet, Cancer Cell, 1:219-27, (2002); Pepper and Skobe, JCB 63:209-13 (2003)). Thus, the lymphatic system plays a vital role in the regulation of fluid, protein, and pressure equilibrium in tissues. By directing leukocytes and antigens from tissues to the lymph nodes, lymphatic vessels also have a key function in immune surveillance. Dysfunction of the lymphatic system results in lymphedema, a chronic and disabling condition for which there are no treatments currently available. Breast cancer treatment is commonly associated with lymphedema, which frequently develops following surgical removal of lymph nodes and radiation therapy.

Lung is a common site for metastasis of many tumors, including common tumors such as breast, colorectal, prostate, bronchial, head-and-neck, and renal cancers (Fidler EJ., Nat Rev Cancer 3:453-8, (2003); Nguyen et al., Nat Rev Cancer 9:274-84 (2009). Pulmonary nodules are the most common manifestation of metastatic cancer in the lungs. They are thought to be derived from tumor emboli which arrest in the lung capillaries and invade into the surrounding lung tissue. Involvement of pulmonary lymphatic vessels with cancer is less commonly diagnosed because of the imaging difficulties. At necropsy, however, metastases that occur via pulmonary lymphatics and bronchial arteries are frequently observed.

Importantly, the involvement of lung lymphatics with cancer is a hallmark of a very aggressive metastatic disease, designated as Lymphangitic Carcinomatosis (Tomashefski and Dail, Dail and Hammar's Pulmonary Pathology (2008); Goldsmith et al., Arch Surg 94:483-8 (1967); Bruce et al., J R Coll Surg Edinb 41:7-13 (1996); Janower et al., Radiology 101:267-73 (1971); Thomas and Lenox., CMAJ 179:338-40 (2008); Das et al., Cancer Res 70:1814-24 (2010)). The prognosis for a patient with this clinical picture is extremely poor; 50% of the patients die within 3 months of diagnosis. Although lymphangitic spread can be caused by any malignant cancer, it most commonly results from tumors originating in the breast, stomach, pancreas, lung, or prostate. This phenomenon is also caused by primary pulmonary carcinoma, especially small cell carcinoma and adenocarcinoma. Because of the extremely aggressive nature of this disease, there is a great need for early diagnosis and treatment. Currently, there is no treatment which improves outcome of patients with Lymphangitic Carcinomatosis.

The molecular mechanisms controlling the growth and function of the lymphatic system are poorly understood. A member of the vascular endothelial growth factor (VEGF) family, VEGF-C, has been identified as a growth factor for lymphatic vessels. VEGF-C is a ligand for the receptor tyrosine kinase VEGFR-3, which is predominantly expressed on lymphatic endothelial cells (Joukov et al., EMBO J 15, 290-98 (1996); Lee et al., Proc Natl Acad Sci USA 93, 1988-92 (1996); Jeltsch et al., Science 276, 1423-5 (1997); Alitalo & Carmeliet, Cancer Cell 1, 219-27 (2002)). VEGF-C also binds to and activates VEGFR-2, which is expressed by lymphatic and by blood endothelium and is also utilized by VEGF-A, a major angiogenesis factor (Joukov et al., EMBO J 16, 3898-911 (1997)). In tumors, VEGFR-3 is expressed by lymphatic endothelial cells and by the subset of blood vessels, but not by tumor cells (Skobe et al., Nature Med 7, 192-8 (2001); Skobe et al., Am J Pathol 159, 893-903 (2001); Roberts et al., Cancer Res 66, 2650-7 (2006); Alitalo et al., Cancer Cell 1, 219-27 (2002); Valtola et al., Am J Pathol 154, 1381-90 (1999); Petrova et al., Cancer Cell 13, 554-6 (2008)). The important role of VEGF-C and VEGFR-3 signaling in developmental and postnatal lymphangiogenesis has been documented. Several studies have also demonstrated that VEGF-C/VEGFR-3 signaling plays a critical role in facilitating spread of metastases from the primary tumor into the lymph nodes. Skobe et al., Nature Med 7(2):192-8 (2001); Mandriota et al., EMBO J 20, 672-82 (2001); Mattila et al., Int J Cancer 98, 946-51 (2002); Krishnan et al., Cancer Res 63, 713-22 (2003); Yanai et al., J Exp Clin Cancer Res 20, 419-28 (2001); Lin et al., Cancer Res 65, 6901-9 (2005); Kawakami et al., Surg Today 35, 131-8 (2005); Chen et al., Cancer Res 65, 9004-11 (2005); Brakenhielm et al., Int J Cancer 121, 2153-61 (2007); Burton et al., Cancer Res 68, 7828-37 (2008).

Several studies have also shown that an increase in lymph node metastases in mice bearing VEGF-C-expressing primary tumors is correlated with an increase in distant metastases. It was demonstrated that VEGF-C increased tumor lymphangiogenesis and cancer spread to the lymph nodes, which was associated with increased metastatic burden in the lung in experimental models of breast cancer, prostate cancer and melanoma. Skobe et al., Nat Med 7, 192-8 (2001); Roberts et al., Cancer Res 66, 2650-7 (2006); Krishnan et al., Cancer Res 63, 713-22 (2003); Lin et al., Cancer Res 65, 6901-9 (2005); Brakenhielm et al., Int J Cancer 121, 2153-61 (2007). Conversely, studies in mouse models of breast cancer, prostate cancer, and melanoma have shown that blocking VEGF-C/VEGFR-3 inhibits tumor lymphangiogenesis and prevents lymph node metastasis in the presence of a primary tumor, and consequently reduces the risk of distant metastasis (Roberts et al., Cancer Res 66, 2650-7 (2006); Krishnan et al., Cancer Res 63, 713-22 (2003); Lin et al., Cancer Res 65, 6901-9 (2005); Chen et al., Cancer Res 65, 9004-11 (2005); Burton et al., Cancer Res 68, 7828-37 (2008)). Based on these findings, VEGF-C/VEGFR-3-mediated lymphangiogenesis would not be considered a target for cancer treatment after the removal of the primary tumor.

Taken together, prior studies examining the effects of blocking VEGF-C and its receptors on tumor metastasis have examined the effects of different antagonists on preventing metastatic spread in the presence of the primary tumor and have not addressed the effects of such antagonists on progression of established distant metastases after removal of the primary tumor.

What is critically needed in the art are compositions and methods for achieving the treatment of established metastatic disease in cases when primary tumors have been removed or are non-resectable.

### SUMMARY OF THE INVENTION

The present invention achieves these and other related needs by providing a new method for inhibiting established tumor metastases in a subject comprising administering to said subject a therapeutically effective amount of one or more VEGFR-3 antagonist(s) and optionally one or more VEGFR-2 antagonist(s).

In a specific embodiment, said antagonist(s) is administered after the eradication or removal of a primary tumor (e.g., by surgery, chemotherapy, radiation therapy, phototherapy, and/or immunotherapy). In another embodiment, said antagonist(s) is administered for inhibition of metastases in a subject in which primary tumor cannot be removed.

In one embodiment, said metastasis is in a distant organ. Non-limiting examples of such distant organs include lung, liver, kidney, peritoneum, and skin.

In one embodiment, said metastasis is in a lymph node.

In another embodiment, said metastasis is Lymphangitic Carcinomatosis.

In a related aspect, the present invention provides a method for inhibiting lymphangiogenesis in a subject comprising administering to said subject a therapeutically effective amount of one or more VEGFR-3 antagonist(s) and optionally one or more VEGFR-2 antagonist(s).

VEGFR-3 antagonist(s) and VEGFR-2 antagonist(s) encompassed by the present invention can be any antagonists. Non-limiting examples of useful antagonists include, e.g., antagonist antibodies and fragments thereof, soluble polypeptides that inhibit the activity of VEGFR-3 or VEGFR-2 (e.g., an extracellular domain of a VEGFR-3 or VEGFR-2 protein or a derivative thereof), small molecule inhibitors (e.g., small molecule inhibitors of kinases and/or signaling pathways relevant for VEGFR-3 and/or VEGFR-2 signal transduction), and inhibitors of VEGFR-3 and/or VEGFR-2 expression (e.g., siRNAs, shRNAs, antisense oligonucleotides, ribozymes, etc.).

In one embodiment, the VEGFR-3 antagonist useful in the methods of the invention is an anti-VEGFR-3 antibody or an antigen-binding portion thereof. In one specific embodiment such VEGFR-3 antagonist is the monoclonal antibody mF4-31C1.

In one embodiment, the VEGFR-2 antagonist useful in the methods of the invention is an anti-VEGFR-2 antibody or an antigen-binding portion thereof. In one specific embodiment such VEGFR-2 antagonist is the monoclonal antibody DC101.

In a specific embodiment, such anti-VEGFR-2 antibody or the anti-VEGFR-3 antibody is capable of binding to an extracellular domain of VEGFR-2 or VEGFR-3, respectively, and is capable of blocking the interaction of VEGF-C, VEGF-D and/or VEGF-A with VEGFR-2 or VEGFR-3. In one embodiment, the antibody is capable of biding to its target (i.e., VEGFR-2 or VEGFR-3) with an affinity of at least about 1×10⁻⁶M, preferably of at least about 1×10⁻⁷ M, more preferably of at least about 1×10⁻⁸ M, most preferably of at least about 1×10⁻⁹M.

The anti-VEGFR-2 antibody or the anti-VEGFR-3 antibody useful in the methods of the present invention can be, e.g., a chimeric antibody, a primatized antibody, a humanized antibody, or an antigen-binding portion thereof. In one embodiment, humanized antibodies of the present invention include one or more CDR from the monoclonal antibody DC101 or one or more CDR from the monoclonal antibody mF4-31C1.

The antigen-binding portion of the antibody useful in the methods of the present invention can be, e.g., an F(ab′)2, a Fab, an Fv, an scFv, or a single domain antibody.

In another embodiment, the VEGFR-2 antagonist or the VEGFR-3 antagonist useful in the methods of the present invention is a soluble polypeptide antagonist. In a specific embodiment, such soluble polypeptide antagonist comprises an extracellular domain of a VEGFR-2 protein or an extracellular domain of a VEGFR-3 protein or an amino acid sequence that is at least 90%, preferably at least 95%, more preferably at least 97%, most preferably at least 99% identical to the extracellular domain of a VEGFR-2 protein or a VEGFR-3 protein. Optionally, one or more soluble peptide antagonist can further comprise a post-translational modification. Non-limiting examples of such post-translational modifications include, e.g., acetylation, carboxylation, glycosylation, phosphorylation, lipidation, acylation, addition of a non-amino acid element (such as, e.g., polyethylene glycol, a lipid, a poly- or mono-saccharide, or a phosphate), and addition of a fusion domain (such as, e.g., polyhistidine, Glu-Glu, glutathione S transferase (GST), thioredoxin, protein A, protein G, an immunoglobulin heavy chain constant region (Fc), a maltose binding protein (MBP), green fluorescent protein (GFP), or an epitope tag). Fusion domains can further comprise a protease cleavage site (such as, e.g., Factor Xa or Thrombin).

In one embodiment, the antagonist(s) of the invention is administered in combination with a radiation treatment or with one or more additional compound(s) useful for inhibiting lymphangiogenesis or metastasis. In a specific embodiment, said additional compound is a chemotherapeutic. In another specific embodiment, said additional compound is an anti-angiogenic compound.

### BRIEF DESCRIPTION OF THE FIGURES

FIG. 1: MDA-MB-435/VEGF-C tumors induce lymphangiogenesis and increase in lymph node size in the tumor draining lymph node. Immunofluorescent staining of LYVE-1 on cryosections of axillary lymph nodes of nu/nu mice shows an increase of medullary lymphatic area in the tumor draining axillary lymph node (C, D) compared to nodes of tumor-free mice (A, B). Nuclei were counterstained with Hoechst. C, Cortex; PC, Paracortex; M, Medulla; Bar:1 mm.

FIG. 2: Computer-based morphometric analysis of axillary lymph node size showing that MDA-MB-435/VEGF-C tumor significantly increased the size of a sentinel lymph node (***p≦0.005).

FIG. 3: Combined blocking of VEGFR-2 and VEGFR-3 most efficiently inhibits lymphangiogenesis and leads to a decrease in size of lymph nodes draining MDA-MB-435/VEGF-C tumors. Immunofluorescent staining for LYVE-1 on cryosections of tumor draining axillary lymph nodes. Nuclei were counterstained with Hoechst. Scale bar: 1 mm.

FIG. 4: Quantification of lymphangiogenesis in tumor draining axillary lymph nodes by computer-based morphometric analysis of LYVE-1 stained cryosections. Results are expressed as percentage lymph node area (*p≦0.05; **p≦0.01; ***p≦0.005).

FIG. 5: Quantification of sentinel lymph node size after anti-VEGFR-2, anti-VEGFR-3 and combined anti-VEGFR-2 and anti-VEGF-3 treatment. MDA-MB-435/VEGF-C tumor induced increase in lymph node size was reduced by blocking VEGFR-2 or VEGFR-3, and most prominently by combination treatment (*p≦0.05; **p≦0.01; ***p≦0.005).

FIG. 6: MDA-MB-435/VEGF-C tumors induce angiogenesis in tumor draining lymph nodes. (A, B) Immunofluorescent staining for CD34 showing microvasculature in the sentinel lymph nodes of tumor-free mice and (C, D) an increase in the density of small blood vessels in the cortex of tumor-draining lymph nodes. (B, D) Higher magnification of the area indicated by the squares. Nuclei were counterstained with Hoechst. C, cortex; PC, paracortex; M, medulla; Bar: 200 μm.

FIG. 7: Quantification of blood vessels in lymph nodes draining MDA-MB-435/VEGF-C tumor. (Upper chart) Tumors induced angiogenesis of the microvasculature (vessel size ranging from 3-60 μm²). (Lower chart) The ratio of blood vessel area per lymph node area remained unchanged, indicating that blood vessel growth parallels increase in lymph node size (***p≦0.005).

FIG. 8: Combined blocking of VEGFR-3 and VEGFR-2 in nu/nu mice bearing MDA-MB-435/VEGF-C tumors most effectively inhibited lymph node angiogenesis. (A, B) Immunofluorescent staining for CD34 showed a high density of microvasculature (MiV) in the cortex (C) of tumor-bearing control mice. (C, D) Blockade of VEGFR-3 by systemic treatment of mice with the specific neutralizing antibodies did not change microvascular density. (E, F) Blockade of VEGFR-2 showed an anti-angiogenic effect in the cortex. (G, H) Dual blocking of VEGFR-3 and VEGFR-2 inhibited lymph node angiogenesis most effectively. Regions indicated by the square are shown in higher magnification. Cell nuclei were counterstained with Hoechst. C, cortex; PC, paracortex; M, medulla; MaV, microvasculature; MiV, microvasculature; HEV, high endothelial venules. Bar in A, C, E, G: 500 μm; in B, D, F, H: 200 μm.

FIG. 9: Neutralization of VEGFR-2 alone or combination of VEGFR-2 and VEGFR-3 inhibited lymph nodes angiogenesis (*p≦0.05; **p≦0.01; ***p≦0.005).

FIG. 10: Different pattern of pulmonary metastases formed by MDA/pcDNA and MDA/VEGF-C cells. Fluorescence stereomicroscopy of thick lung sections (50 m) showing distribution of MDA/pcDNA (A, B) and MDA/VEGF-C (D, E) metastases (red) in the lung. (C, F) Confocal analysis of a metastatic lesion formed by MDA/pcDNA (C) or MDA/VEGF-C (F) cells. Note tight association of MDA/VEGF-C metastases with the airways. (G) Comparison of the average size of metastatic foci. (H) Comparison of the size of metastatic foci in the lung parenchyma vs. peribronchial area. b, bronchi. Scale bar: 50 μm.

FIG. 11: Pulmonary metastases formed by MDA/VEGF-C cells are associated with the airways. H&E-stained lung sections bearing MDA/pcDNA (A-D) or MDA/VEGF-C (E-H) metastases. (A-D) Typical MDA/pcDNA metastatic foci localized in the peripheral lung parenchyma distant from the small (A, B) and large (C, D) bronchi. (E-H) MDA/VEGF-C metastases surrounding small (E, F) and large (G, H) bronchi. b, bronchi; t, tumor. Scale bar:

FIG. 12. Pulmonary tumor emboli are characteristic of MDA/VEGF-C metastases. (A-C) Immunohistochemistry of paraffin-embedded lung sections for a-smooth muscle actin (α-SMA) showing MDA/VEGF-C tumor cells (t) in large and small pulmonary arteries (arrows) associated with the airways (b, bronchi). (D-F) α-SMA staining of lungs with MDA/pcDNA metastases. Note that MDA/pcDNA tumor cells are neither seen in pulmonary arteries (arrows) (D, E) nor in veins (v) (F). (G) Confocal image of tumor emboli (red, RFP) in the pulmonary artery. (H) Immunofluorescent staining for α-SMA (red) showing MDA/VEGF-C tumor embolus (t, green) in a small artery (arrow) adjacent to bronchus (b). Bronchial smooth muscle also stains positive for α-SMA. (I) Staining for blood endothelial marker CD34 (purple) showing small artery (arrow) with MDA/VEGF-C tumor embolus, as well as pulmonary capillaries. (J) Large MDA/VEGF-C metastatic lesion (t, green) involving pulmonary arteries (arrows) immunostained with α-SMA, and the surrounding peribronchial interstitium. (K) Typical MDA/pcDNA metastatic foci containing only a few scattered α-SMA-positive cells. (L, M) MDA/pcDNA metastasis adjacent to the pulmonary vessel (arrow) stained with α-SMA (red) (L) and CD34 (purple) (M). Note that MDA/pcDNA tumor cells are not seen inside of the large blood vessel. (N, O) MDA/pcDNA tumor cells (green, arrowheads) localized within pulmonary capillaries stained with VWF (red). Immunofluorescent staining was analyzed by confocal microscopy. Scale bars: 200 μm (A-F); 50 μm (G-M); 25 μm (N,O). Blue, Hoechst nuclear stain; b, bronchi; t, tumor cells. Data are representative of at least 3 experiments.

FIG. 13. Pulmonary lymphatic vessels are dilated throughout the lungs VEGF-C-expressing metastases. (A-C) Immunofluorescent staining for VEGFR-3 (red, arrows) showing lymphatic vessels adjacent to the airways in the lung of normal, healthy mouse. (D-G) Immunohistochemical staining for SMA a-actin (D) and LYVE-1 (E-G), showing enlarged lymphatic in the lungs with MDA/VEGF-C metastases. Nuclei are stained with Hoechst (blue). Scale bar: 100

FIG. 14. Evolution of lymphangiogenesis associated with expansion of metastases. (A-C) Immunofluorescence analysis showing small MDA/VEGF-C metastatic nodule (t, green) next to the lymphatic vessel (VEGFR-3, red) in the peribronchial region (b, bronchus). Note that during the early stages of metastases lymphatics are not changed in number or appearance. (D-F) Dilated lymphatic vessels (arrows) surround larger metastatic lesions and new lymphatics line the edge of metastasis. (G-I) Large metastatic lesion becomes infiltrated with lymphatics and many lymphatics contain tumor cells. (J-L) Drastic expansion of the lymphatic network and extreme dilation of lymphatics (arrows) is associated with very large metastases. Note that lymphatic vessels radically change in number and in appearance, but that metastases and the associated lymphatics always localize in proximity of the airways. (M, N) Quantification of lymphangiogenesis performed by measuring lymophatic vessel area. Lymphatics were visualized by immunofluorescent staining for VEGFR-3 (red). MDA/VEGF-C metastases are GFP-labeled (green). Nuclei are stained with Hoechst (blue). Scale bar: 100 μm.

FIG. 15: Lung metastases expressing VEGF-C induce proliferation of lymphatic endothelial cells. (A) MDA/VEGF-C metastases present inside the large lymphatic vessels showing many proliferating LECs. (B, C) magnification of the boxed areas in A. Mouse lung tissue was immunostained with anti-mouse Ki67 and anti-mouse VEGFR-3. t, tumor cells; arrows, Ki67-labeled LECs. Scale bars: A, 100 μm; B, C, 25 μm. (D) Quantification of proliferating LECs, identified as mKi67+/VEGFR-3+ cells within the lymphatic vessel wall. n=3 mice. ***P<0.001.

FIG. 16: Intravascular localization of tumor metastases in the peri-bronchial space (A-C) Immunohistochemical staining for smooth muscle α-actin (A, B) and LYVE-1 (C), showing large lymphatic vessels associated to the bronchiole containing densely packed tumor cells. (D-F) Immunofluorescent staining for smooth muscle α-actin (D) and podoplanin (E), showing large collecting lymphatic vessel adjacent to the bronchi containing large tumor mass (F). Tumor cells are GFP-labeled (green).

FIG. 17: Confocal analysis and three-dimensional reconstruction of a metastatic lesion expressing VEGF-C inside the lymphatic vessels in the lung. (A) Cross-section and (B) longitudinal projection of a lung lymphatic vessel immunostained for LYVE-1 (green) containing tumor cell clusters (RFP, red).

FIG. 18. VEGF-C promotes secondary metastases to the thoracic lymph nodes. (A) Combined bright field and fluorescence stereomicroscopy of the mouse thoracic cavity showing lymph node positive for GFP-labeled MDA/VEGF-C metastasis (green). (B, C) Ex vivo analysis of the thoracic lymph nodes by stereomicroscopy showing 3 lymph nodes positive for metastases (green). (D) Tumor cells in the sub-capsular sinus of the lymph node (arrow). (Inset) Lymphatic sinus is immunostained for podoplanin (red), nuclei are stained with Hoechst (blue). (E) H&E staining of lymph node section with metastases (t) infiltrating into the node. (Inset) Higher magnification of trachea adjacent to the lymph node, showing localization and relative size of the thoracic node. Scale bar: 200 μm.

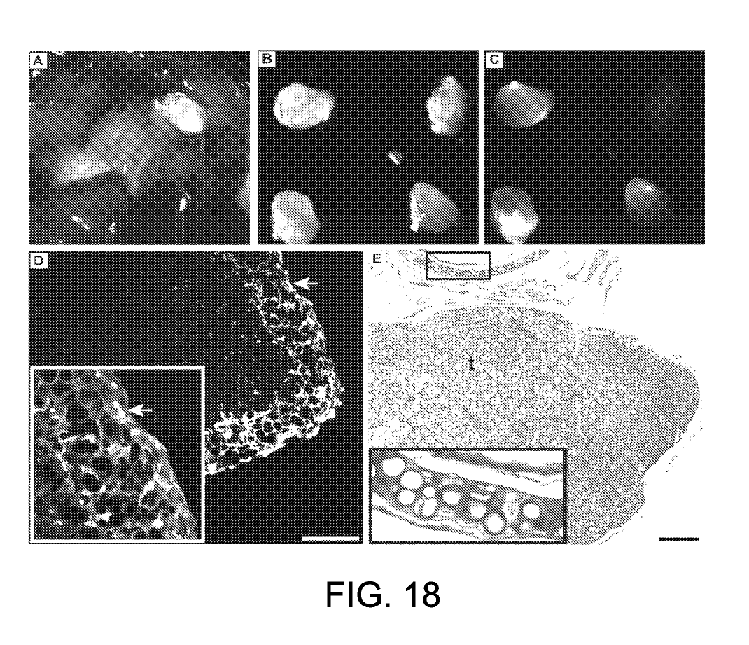

FIG. 19. Inhibition of VEGFR-3 signaling with function-blocking antibody alters the phenotype of peribronchial metastases. Blocking antibody was administered at 800 μg/mouse every second day after the removal of the primary tumor (MDA/VEGF-C) and metastases were analyzed after six weeks of treatment. (A) H&E showing metastases (t, tumor) in the peribronchial area (b, bronchi). (B) Immunohistochemical staining for lymphatic marker LYVE-1 (arrow points to a lymphatic vessel), shows that the metastases are not present in the lymphatic vessels, as well as complete absence of lymphangiogenesis. (C) Staining for smooth muscle α-actin (SMA-α, arrows), showing that peribronchial metastases in the proximity of the airways and the large pulmonary vessels are not presenting as pulmonary artery tumor embolism. (D) Immunofluorescent staining showing metastases (t) adjacent to bronchi (b) which are neither present in the pulmonary artery nor in the lymphatic vessels. Nuclei are stained with Hoechst. Compare with FIG. 16, which shows typical phenotype of VEGF-C expressing metastases which grow in the lymphatic vessels and present as pulmonary artery tumor emboli.

FIG. 20. (A) Inhibition of VEGFR-3 signaling with the function-blocking antibody suppresses growth of metastases after the removal of the primary tumor (MDA/VEGF-C). Data was obtained by bioluminescent imaging of the lungs ex vivo. In control, the highest value (4.8×10⁸) was not plotted on the chart to better show the distribution of the values; however, this value has been included in the calculation of the average values. (B) Metastases in the anti-VEGFR-3-treated group commonly present in the lung parenchyma, similar to the pattern of slowly progressing metastases which do not express VEGF-C.

### DETAILED DESCRIPTION OF THE INVENTION

As specified in the Background section, to date, the lymphatic system has been thought to serve solely as a pathway for dissemination of cancer from the primary tumor into the lymph nodes and targeting of lymphatic vessels has been viewed as an approach to inhibit spread of cancer cells from the primary tumor to the regional lymph nodes, and consequently prevent distant spread. In a clinical setting, however, a major challenge is a treatment of an established metastatic disease after the primary tumor has been surgically removed or eradicated otherwise or is unresectable.

The present invention is based on the unexpected discovery that VEGF-C signaling is important for metastatic spread and growth even in the absence of the primary tumor. As disclosed herein, blocking lymphangiogenesis by targeting VEGF-C and its receptors can be effective not only for prevention, but also for treatment of established metastatic disease. The present invention challenges the existing paradigm that the lymphatic system plays a role in metastasis primarily as a pathway for spread from the primary tumor into the regional lymph nodes. As shown herein, VEGF-C-mediated lymphangiogenesis at the secondary site (e.g., in the lung) promotes aggressive behavior of metastases which have already arrived to the distant organ. As further disclosed herein, newly formed lymphatic vessels in different organs serve as a niche in which metastases rapidly grow. By demonstrating that lymphangiogenesis plays an important role in advancing not only early, but also the late steps of the metastatic process, the present invention provides new uses for anti-lymphangiogenesis inhibitors in treatment of cancer. In a clinical setting, a major challenge is treatment of established metastatic disease after the primary tumor has been surgically removed, eradicated by other means, or is unresectable. The present invention provides a novel method for treating such established metastatic disease by blocking lymphangiogenesis using antagonists of VEGF-C receptors, VEGFR-3 and VEGFR-2.

Specifically, the present invention provides a method for inhibiting an established tumor metastasis in a subject comprising administering to said subject a therapeutically effective amount of one or more VEGFR-3 antagonist(s) and optionally one or more VEGFR-2 antagonist(s). In a related embodiment, the invention provides a method for inhibiting lymphangiogenesis in a subject with a metastatic disease comprising administering to said subject a therapeutically effective amount of one or more VEGFR-3 antagonist(s) and optionally one or more VEGFR-2 antagonist(s).

As used herein, the term “inhibiting an established tumor metastasis” refers to decreasing the size and/or rate of growth of a metastasis which has been already established.

Metastases encompassed by the present invention include metastases in lymph nodes (regional metastases) and distant organs (systemic metastases).

As used herein, the term “lymphangiogenesis” refers to growth of new lymphatic vessels.

As used herein, the term “therapeutically effective” applied to dose or amount refers to that quantity of a VEGFR-3 antagonist(s) and/or VEGFR-2 antagonist(s) or a pharmaceutical composition containing such antagonist(s) that is sufficient to result in a desired therapeutic activity upon administration to a subject in need thereof, or sufficient to reduce or eliminate at least one symptom of the disease being treated.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “subject” means any animal, including mammals. In particular, the term may refer to a human, a non-human primate, a bovine, an ovine, an equine, a porcine, a canine, a feline, or a rodent (mouse or rat).

As used in this specification and the appended claims, the singular forms “a,” “an,” and “the” include plural references unless the context clearly dictates otherwise.

As disclosed herein, the pattern of metastatic spread to the lungs observed with VEGF-C expressing cells in a MDA-MB-435/VEGF-C mouse model for breast cancer closely resembles Lymphangitic Carcinomatosis aggressive metastatic phenotype in human cancer patients. As described in greater detail below, tumors that do not express VEGF-C do not show any evidence of lymphatic involvement in the lungs, while VEGF-C facilitates lung lymphangiogenesis, tumor cell entry into the lung lymphatics and growth within, creating a niche for tumor expansion within the lung as well as a route for dissemination to the thoracic lymph nodes. Thus, as demonstrated herein, VEGF-C expression by tumor cells drastically changes the pattern of metastatic disease and facilitates disease progression.

The data presented herein demonstrate for the first time that the VEGF-C/VEGFR-3 pathway and the lymphatic vessels are targets for the treatment of established metastatic disease, after the removal of the primary tumor. Interestingly, Lymphangitic Carcinomatosis is an aggressive disease that has been observed in association with many common metastatic cancers such as breast, gastric, pancreatic, prostate cancer and others. Primary lung cancer can also present in the form of Lymphangitic Carcinomatosis, suggesting that targeting of VEGF-C/VEGFR-3 in lung cancer could be a treatment option for slowing the progression of lung cancer.

Clinically, Lymphangitic Carcinomatosis is characterized by the presence of malignant cells in the lymphatic vessels localized in the peri-bronchovascular area, in the interlobular septa, and in the centri-lobular region. Associated pleural involvement is common. Edema, resulting from blockage of lymphatic drainage and a desmoplastic reaction, are common and can contribute to interstitial thickening. Hilar and mediastinal lymphadenopathy are present in 20-40% of patients, and pleural effusions are present in 30-50% of patients. The similarities between the clinical picture of Lymphangitic Carcinomatosis and pulmonary metastatic disease induced by VEGF-C are presented in the Table 1.

TABLE 1

| | Lymphangitic | VEGF-C |
|---|---|---|
| | Carcinomatosis | expression |
| Lung architecture | Normal | Normal |
| Centri-lobular LV involvement | Yes | Yes |
| Peri-bronchovascular LV involvement | Yes | Yes |
| Interlobular septa LV involvement | Yes | ND |
| Pulmonary artery involvement | Yes | Yes |
| Rapid progression of the disease | Yes | Yes |
| Metastases in the pleura | Yes | Yes |
| Metastases in the thoracic nodes | Yes | Yes |

LV, lymphatic vessels; ND, no determined

### VEGFR-2 and VEGFR-3 Antagonists and Compositions Thereof

As indicated above, the present invention provides a method for treating established metastases, by administering one or more VEGFR-3 antagonist(s) and optionally one or more VEGFR-2 antagonist(s). Thus, provided herein are compositions comprising one or more VEGFR-3 antagonist(s), one or more VEGFR-2 antagonist(s), or combinations of one or more VEGFR-3 antagonist(s) and one or more VEGFR-2 antagonist(s).

VEGFR-3 antagonist(s) and VEGFR-2 antagonist(s) encompassed by the present invention can be any antagonists. Non-limiting examples of useful antagonists include, e.g., antagonist antibodies and fragments thereof, soluble polypeptides that inhibit the activity of VEGFR-3 or VEGFR-2 (e.g., an extracellular domain of a VEGFR-3 or VEGFR-2 protein or a derivative thereof), small molecule inhibitors (e.g., small molecule inhibitors of kinases and/or signaling pathways relevant for VEGFR-3 and/or VEGFR-2 signal transduction [see, e.g., Zhang et al., 2009, Nature 9:28-39; Krishnan et al., Cancer Res., 2003, 63:713-22]), and inhibitors of VEGFR-3 and/or VEGFR-2 expression (e.g., siRNAs, shRNAs, antisense oligonucleotides, ribozymes, etc.).

VEGFR-2 and VEGFR-3 Antibody Antagonists

The present disclosure provides, in part, compositions comprising one or more anti-VEGFR-3 (VEGFR-3 is also known as FLT-4) antagonist antibody, compositions comprising one or more anti-VEGFR-2 (VEGFR-2 is also known as FLK-1 and KDR) antagonist antibody, or a combination of anti-VEGFR-3 and anti-VEGFR-2 antagonist antibodies, and/or antigen binding portions of such antagonist antibodies, that inhibit one or more VEGFR-3- and/or one or more VEGFR-2-mediated functions, such as a ligand (e.g., VEGF-C, VEGF-D, and/or VEGF-A) binding and/or a signaling activity (e.g., VEGFR-3 or VEGFR-2 dimerization and/or transphosphorylation). Typically, the antagonist antibody binds to an extracellular domain of VEGFR-3 or VEGFR-2.

In one embodiment, antibodies are raised against an isolated and/or recombinant mammalian VEGFR-2 or VEGFR-3, or a portion thereof, or against a host cell that expresses recombinant mammalian VEGFR-2 or VEGFR-3. In certain aspects, antibodies of the present disclosure specifically bind to an extracellular domain of a VEGFR-2 or VEGFR-3 protein. As shown in the Examples below, the combination of anti-VEGFR-2 monoclonal antibody DC101 (ImClone Systems) and anti-VEGFR-3 monoclonal antibody mF4-31C1 (ImClone Systems) is able to inhibit lymph node lymphangiogenesis and growth of metastases expressing VEGF-C.

Anti-VEGFR-2 antibodies useful in the methods of the invention will typically be specific for VEGFR-2 and anti-VEGFR-3 antibodies useful in the methods of the invention will typically be specific for VEGFR-3, with minimal binding to other members of the VEGFR families. Optionally, the antibodies bind to VEGFR-2 and VEGFR-3 with an affinity of at least about 1×10⁻⁶ M, preferably at least about 1×10⁻⁷ M, more preferably at least about 1×10⁻⁸ M, most preferably at least about 1×10⁻⁹ M, or even less.

The term “antibody” as used herein is intended to include monoclonal and polyclonal antibodies as well as any full length immunoglobulin chains, including chimeric and humanized forms. An “isolated antibody” is an antibody that is substantially purified or produced so as to be free of other species of antibodies that bind to the same target. Monoclonal antibodies and most recombinant antibody forms are typically isolated prior to administration to a subject. Antigen binding portions of an antibody include, e.g., F(ab′)₂, Fab, Fv, scFv, and single domain antibodies.

In certain embodiments, single chain antibodies, and chimeric, humanized, or primatized (CDR-grafted) antibodies, as well as chimeric or CDR-grafted single chain antibodies, comprising portions derived from different species, are also encompassed by the present invention as antigen binding portions of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger et al., WO 86/01533; Neuberger et al., European Patent No. 0,194,276 B1; Winter, U.S. Pat. No. 5,225,539; and Winter, European Patent No. 0,239,400 B1. See also, Newman et al., BioTechnology 10:1455-1460 (1992), regarding primatized antibody and Ladner et al., U.S. Pat. No. 4,946,778 and Bird et al., Science 242:423-426 (1988)), regarding single chain antibodies.

In addition, functional fragments of antibodies, including fragments of chimeric, humanized, primatized, or single chain antibodies can also be produced. Functional fragments of the subject antibodies retain at least one binding function and/or modulation function of the full-length antibody from which they are derived. Preferred functional fragments retain an antigen binding function of a corresponding full-length antibody (e.g., specificity for VEGFR-2 or VEGFR-3). Certain preferred functional fragments retain the ability to inhibit one or more functional characteristics of a VEGFR, such as a ligand (e.g., VEGF-C, VEGF-D, VEGF-A) binding activity and/or a signaling activity.

For example, antibody fragments capable of binding to a VEGFR-2 or VEGFR-3 receptor or portion thereof, including, but not limited to, Fv, Fab, Fab′ and F(ab′)₂ fragments are encompassed by the present disclosure. Such fragments can be produced by enzymatic cleavage or by recombinant techniques. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH1 domain and hinge region of the heavy chain.

The term “humanized immunoglobulin” as used herein refers to an immunoglobulin comprising portions of immunoglobulins of different origin, wherein at least one portion is of human origin. Accordingly, the present disclosure relates to a humanized immunoglobulin having binding specificity for a VEGFR-2 or VEGFR-3, wherein the immunoglobulin comprises an antigen binding region of nonhuman origin (e.g., rodent) and at least a portion of an immunoglobulin of human origin (e.g., a human framework region, a human constant region or portion thereof). For example, the humanized antibody can comprise portions derived from an immunoglobulin of nonhuman origin with the requisite specificity, such as a mouse, and from immunoglobulin sequences of human origin (e.g., a chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain).

Another example of a humanized immunoglobulin of the present disclosure is an immunoglobulin containing one or more immunoglobulin chains comprising a CDR of nonhuman origin (e.g., one or more CDRs derived from an antibody of nonhuman origin) and a framework region derived from a light and/or heavy chain of human origin (e.g., CDR-grafted antibodies with or without framework changes). In one embodiment, the humanized immunoglobulin can compete with murine monoclonal antibody for binding to a VEGFR polypeptide. Chimeric or CDR-grafted single chain antibodies are also encompassed by the term humanized immunoglobulin.

In certain embodiments, anti-idiotypic antibodies are also provided. Anti-idiotypic antibodies recognize antigenic determinants associated with the antigen-binding site of another antibody. Anti-idiotypic antibodies can be prepared against a second antibody by immunizing an animal of the same species, and preferably of the same strain, as the animal used to produce the second antibody. See, e.g., U.S. Pat. No. 4,699,880. In one embodiment, antibodies are raised against VEGFR-2 or VEGFR-3 or a portion thereof, and these antibodies are used in turn to produce an anti-idiotypic antibody. The anti-idiotypic antibodies produced thereby can bind compounds which bind receptor, such as ligands of receptor function, and can be used in an immunoassay to detect or identify or quantify such compounds. Such an anti-idotypic antibody can also be an inhibitor of VEGFR-2 or VEGFR-3 function, although it does not bind receptor itself. Such an anti-idotypic antibody is also referred to, herein, as an antagonist antibody.

In certain aspects, the present disclosure provides the hybridoma cell lines, as well as the monoclonal antibodies produced by these hybridoma cell lines. Such cell lines can be fused with other cells (such as suitably drug-marked human myeloma, mouse myeloma, human-mouse heteromyeloma, or human lymphoblastoid cells) to produce additional hybridomas, and thus provide for the transfer of the genes encoding the monoclonal antibodies. In addition, the cell lines can be used as a source of nucleic acids encoding the anti-VEGFR-2 and/or anti-VEGFR-3 immunoglobulin chains, which can be isolated and expressed (e.g., upon transfer to other cells using any suitable technique (see, e.g., Cabilly et al., U.S. Pat. No. 4,816,567; Winter, U.S. Pat. No. 5,225,539)). For instance, clones comprising a rearranged anti-VEGFR light or heavy chain can be isolated (e.g., by PCR) or cDNA libraries can be prepared from mRNA isolated from the cell lines, and cDNA clones encoding an anti-VEGFR immunoglobulin chain can be isolated. Thus, nucleic acids encoding the heavy and/or light chains of the antibodies or portions thereof can be obtained and used in accordance with recombinant DNA techniques for the production of the specific immunoglobulin, immunoglobulin chain, or variants thereof (e.g., humanized immunoglobulins) in a variety of host cells or in an in vitro translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host cell by an appropriate method (e.g., transformation, transfection, electroporation, or infection), such that the nucleic acid is operably linked to one or more expression control elements (e.g., in the vector or integrated into the host cell genome). For production, host cells can be maintained under conditions suitable for expression (e.g., in the presence of inducer, suitable media supplemented with appropriate salts, growth factors, antibiotic, nutritional supplements, etc.), whereby the encoded polypeptide is produced. If desired, the encoded protein can be recovered and/or isolated (e.g., from the host cells or medium). It will be appreciated that the method of production encompasses expression in a host cell of a transgenic animal (see, e.g., WO92/03918).

Preparation of Immunizing Antigen, and Polyclonal and Monoclonal Antibody production, can be performed as described herein, or using other suitable techniques. A variety of methods have been described. See, e.g., Kohler et al., Nature 256:495-497 (1975) and Eur. J. Immunol. 6:511-519 (1976); Milstein et al., Nature 266:550-552 (1977); Koprowski et al., U.S. Pat. No. 4,172,124; Harlow and Lane, “Antibodies: A Laboratory Manual,” (Cold Spring Harbor Laboratory: Cold Spring Harbor, N.Y., 1988); and “Current Protocols In Molecular Biology,” (Ausubel et al., Eds.; John Wiley & Sons: New York, N.Y., 1991). Generally, a hybridoma can be produced by fusing a suitable immortal cell line (e.g., a myeloma cell line such as SP2/0) with antibody producing cells. The antibody producing cell, preferably those of the spleen or lymph nodes, are obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library, or which rely upon immunization of transgenic animals (e.g., mice) capable of producing a full repertoire of human antibodies. See, e.g., Jakobovits et al., Proc. Natl. Acad. Sci. USA 90:2551-2555 (1993); Jakobovits et al., Nature 362:255-258 (1993); Lonberg et al., U.S. Pat. No. 5,545,806; Surani et al., U.S. Pat. No. 5,545,807.

Immunogens derived from a VEGFR-2 or VEGFR-3 polypeptide (or an antigenic fragment thereof which is capable of eliciting an antibody response) can be used to immunize a mammal, such as a mouse, a hamster or rabbit. See, e.g., “Antibodies: A Laboratory Manual” (Ed. by Harlow and Lane, Cold Spring Harbor Press, 1988). Techniques for conferring immunogenicity on a protein or peptide include conjugation to carriers or other techniques well known in the art. An immunogenic portion of a VEGFR-2 or VEGFR-3 polypeptide can be administered in the presence of adjuvant. The progress of immunization can be monitored by detection of antibody titers in plasma or serum. Standard ELISA or other immunoassays can be used with the immunogen as antigen to assess the levels of antibodies. In one embodiment, antibodies of the disclosure are specific for the extracellular portion of the VEGFR-2 or VEGFR-3 protein or fragments thereof.

Following immunization of an animal with an antigenic preparation of a VEGFR-2 or VEGFR-3 polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature 256:495-497 (1975)), the human B cell hybridoma technique (Kozbar et al., Immunology Today 4:72 (1983)), and the EBV-hybridoma technique to produce human monoclonal antibodies (Cole et al., “Monoclonal Antibodies and Cancer Therapy,” Alan R. Liss, Inc. pp. 77-96 (1985)). Hybridoma cells can be screened immunochemically for production of antibodies specifically reactive with a VEGFR-2 or VEGFR-3 polypeptide and monoclonal antibodies isolated from a culture comprising such hybridoma cells.

In certain embodiments, antibodies of the present disclosure can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments.

In certain embodiments, antibodies of the present disclosure are further intended to include bispecific, single-chain, and chimeric and humanized molecules having affinity for an VEGFR-2 or VEGFR-3 polypeptide conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. Methods of generating these antibodies are known in the art. See, e.g., Cabilly et al., U.S. Pat. No. 4,816,567; Cabilly et al., European Patent No. 0,125,023 B1; Queen et al., European Patent No. 0,451,216 B1; Boss et al., U.S. Pat. No. 4,816,397; Boss et al., European Patent No. 0,120,694 E1; Neuberger et al., WO 86/01533; Neuberger et al., European Patent No. 0,194,276 B1; Winter, U.S. Pat. No. 5,225,539; Winter, European Patent No. 0,239,400 B1; and Padlan et al., European Patent Application No. 0,519,596 A1. See, also, Ladner et al., U.S. Pat. No. 4,946,778; Huston, U.S. Pat. No. 5,476,786; and Bird et al., Science 242: 423-426 (1988)).

Such humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see, e.g., Kamman et al., Nucl. Acids Res., 17:5404 (1989)); Sato et al., Cancer Research 53:851-856 (1993); Daugherty et al., Nucleic Acids Res. 19(9):2471-2476 (1991); and Lewis and Crowe, Gene 101:297-302 (1991)). Using these or other suitable methods, variants can also be readily produced. In one embodiment, cloned variable regions can be mutagenized, and sequences encoding variants with the desired specificity can be selected (e.g., from a phage library; see, e.g., Krebber et al., U.S. Pat. No. 5,514,548; and Hoogenboom et al., WO 93/06213).

In certain embodiments, antibodies of the present disclosure are monoclonal antibodies. A method for generating a monoclonal antibody that binds specifically to a VEGFR-2 or a VEGFR-3 polypeptide may comprise administering to a mouse an amount of an immunogenic composition comprising the VEGFR polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the VEGFR polypeptide. Once obtained, a hybridoma can be propagated in a cell culture, optionally in culture conditions where the hybridoma-derived cells produce the monoclonal antibody that binds specifically to VEGFR-2 or VEGFR-3. The monoclonal antibody may be isolated from the cell culture.

In addition, the techniques used to screen antibodies in order to identify a desirable antibody may influence the properties of the antibody obtained. For example, to obtain antibodies binding to the extracellular domain of the corresponding VEGFR, it may be desirable to screen for antibodies that bind to cells that express the antigen of interest (e.g., by fluorescence activated cell sorting). A variety of techniques are available for testing antibody:antigen interactions to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc., Gaithersburg, Md.), western blots, immunoprecipitation assays, and immunohistochemistry.

In certain embodiments, the antibodies or antigen binding fragments of the antibodies can be labeled or unlabeled for diagnostic purposes. Typically, diagnostic assays entail detecting the formation of a complex resulting from the binding of an antibody to VEGFR-2 or VEGFR-3. The antibodies can be directly labeled with, for example, a radionuclide, a fluorophore, an enzyme, an enzyme substrate, an enzyme cofactor, an enzyme inhibitor, and a ligand (e.g., biotin or a hapten). Numerous appropriate immunoassays are known to the skilled artisan (see, e.g., U.S. Pat. Nos. 3,817,827; 3,850,752; 3,901,654; and 4,098,876).

VEGFR-2 and VEGFR-3 Soluble Peptide Antagonists

The present invention also contemplates a wide variety of soluble polypeptides that inhibit the activity of VEGFR-3 or VEGFR-2. Such soluble polypeptide antagonists include, for example, an extracellular domain of a VEGFR-3 or VEGFR-2 protein. Typically, such soluble polypeptides are capable of binding with high affinity to a ligand such as, for example, VEGF-C, VEGF-D or VEGF-A. In a specific embodiment, the soluble polypeptide comprises a globular domain of a VEGFR-2 or VEGRF-3 protein. As used herein, soluble polypeptides include fragments, functional variants, and modified forms of VEGFR-3 or VEGFR-2 soluble polypeptides. These fragments, functional variants, and modified forms of soluble polypeptides antagonize the function of VEGFR-3, VEGFR-2, or both.

Isolated fragments of these soluble polypeptides can be obtained by screening polypeptides recombinantly produced from the corresponding fragment of the nucleic acid encoding a VEGFR-2 or VEGFR-3 soluble polypeptide. In addition, fragments can be chemically synthesized using techniques known in the art such as, e.g., conventional Merrifield solid phase f-Moc or t-Boc chemistry. The fragments can be produced (recombinantly or by chemical synthesis) and tested to identify those peptidyl fragments that can function to inhibit function of VEGFR-3 or VEGFR-2 or both.

In certain embodiments, a functional variant of a VEGFR-2 or VEGFR-3 soluble polypeptide comprises an amino acid sequence that is at least 90%, preferably at least 95%, more preferably at least 97%, most preferably at least 99% identical to the extracellular domain of VEGFR-2 or VEGFR-3.

In certain embodiments, the present invention contemplates making functional variants by modifying the structure of a soluble polypeptide antagonist for such purposes as enhancing therapeutic efficacy or stability (e.g., ex vivo shelf life and resistance to proteolytic degradation in vivo). Modified soluble polypeptides can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

The present disclosure further contemplates a method of generating sets of combinatorial mutants of the VEGFR-2 or VEGFR-3 soluble polypeptides, as well as truncation mutants, and is especially useful for identifying functional variant sequences. The purpose of screening such combinatorial libraries may be to generate, for example, soluble polypeptide variants that can act as antagonists of VEGFR-2 or VEGFR-3. Combinatorially-derived variants can be generated that have a selective potency relative to a naturally occurring soluble polypeptide. Such variant proteins, when expressed from recombinant DNA constructs, can be used in gene therapy protocols. Likewise, mutagenesis can give rise to variants that have in vivo half-lives dramatically different than the corresponding wild-type soluble polypeptide. For example, the altered protein can be rendered either more stable or less stable to proteolytic degradation or other cellular process that result in its destruction or inactivation. A short half-life can give rise to more transient biological effects and, when part of an inducible expression system, can allow tighter control of recombinant soluble polypeptide levels within a cell.

There are many ways by which a library of potential homologs can be generated from a degenerate oligonucleotide sequence. Chemical synthesis of a degenerate gene sequence can be carried out in an automatic DNA synthesizer, and the synthetic genes can then be ligated into an appropriate gene for expression. A degenerate set of genes provides, in one mixture, all of the sequences encoding the desired set of potential soluble polypeptide sequences. The synthesis of degenerate oligonucleotides is well known in the art (see, e.g., Narang, Tetrahedron 39:3 (1983); Itakura et al., “Recombinant DNA,” (Proc. 3rd Cleveland Sympos. Macromolecules, ed. A G Walton, Amsterdam: Elsevier pp 273-289 (1981)); Itakura et al., Annu. Rev. Biochem. 53:323 (1984); Itakura et al., Science 198:1056 (1984); and Ike et al., Nucleic Acid Res. 11:477 (1983). Such techniques have been employed in the directed evolution of other proteins (see, e.g., Scott et al., Science 249:386-390 (1990); Roberts et al., Proc. Natl. Acad. Sci. U.S.A. 89:2429-2433 (1992); Devlin et al., Science 249:404-406 (1990); Cwirla et al., Proc. Natl. Acad. Sci. U.S.A. 87:6378-6382 (1990); and U.S. Pat. Nos. 5,223,409, 5,198,346, and 5,096,815).

Alternatively, other forms of mutagenesis can be utilized to generate a combinatorial library. For example, soluble polypeptide variants, which retain anti-VEGFR-2 or anti-VEGFR-3 antagonist activity, can be generated and isolated from a library by screening using, for example, alanine scanning mutagenesis and the like (Ruf et al., Biochemistry 33:1565-1572 (1994); Wang et al., J. Biol. Chem. 269:3095-3099 (1994); Balint et al., Gene 137:109-118 (1993); Grodberg et al., Eur. J. Biochem. 218:597-601 (1993); Nagashima et al., J. Biol. Chem. 268:2888-2892 (1993); Lowman et al., Biochemistry 30:10832-10838 (1991); and Cunningham et al., Science 244:1081-1085 (1989)), by linker scanning mutagenesis (Gustin et al., Virology 193:653-660 (1993); Brown et al., Mol. Cell Biol. 12:2644-2652 (1992); and McKnight et al., Science 232:316 (1982)); by saturation mutagenesis (Meyers et al., Science 232:613 (1986)); by PCR mutagenesis (Leung et al., Methods Cell. Mol. Biol. 1:11-19 (1989)); or by random mutagenesis, including chemical mutagenesis, (Miller et al., “A Short Course in Bacterial Genetics,” (Cold Spring Harbor Laboratory Press, Cold Spring Harbor, N.Y. (1992); and Greener et al., Strategies in Mol. Biol. 7:32-34 (1994)). Linker scanning mutagenesis, particularly in a combinatorial setting, is an attractive method for identifying truncated (bioactive) forms of the subject soluble polypeptide.

A wide range of techniques are known in the art for screening gene products of combinatorial libraries made by point mutations and truncations, and for screening cDNA libraries for gene products having a certain property. Such techniques may be adapted for rapid screening of the gene libraries generated by the combinatorial mutagenesis of the subject soluble VEGFR-2 and VEGFR-3 polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, the subject soluble polypeptides of the disclosure include a small molecule such as a peptide and a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of VEGFR-2 or VEGFR-3 soluble polypeptides.

In certain embodiments, the soluble polypeptides of the disclosure may further comprise post-translational modifications. Such modifications include, but are not limited to, acetylation, carboxylation, glycosylation, phosphorylation, lipidation, and acylation. As a result, the modified soluble polypeptides may contain non-amino acid elements, such as polyethylene glycols, lipids, poly- or mono-saccharide, and phosphates. Effects of such non-amino acid elements on the functionality of a soluble polypeptide can be tested, e.g., by its ability to inhibit VEGFR-2 or VEGFR-3 ligand (e.g., VEGF-C, VEGF-D, VEGF-A) binding and/or signaling function.

In certain aspects, functional variants or modified forms of the subject soluble polypeptides include fusion proteins having at least a portion of the soluble polypeptide and one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography.

For the purpose of affinity purification, relevant matrices for affinity chromatography, such as glutathione-, amylase-, and nickel- or cobalt-conjugated resins are used. Another fusion domain well known in the art is green fluorescent protein (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the soluble polypeptides contain one or more modifications that are capable of stabilizing the soluble polypeptides. For example, such modifications enhance the in vivo (e.g., circulatory) half-life of the soluble polypeptides.

Soluble polypeptides (unmodified or modified) can be produced by a variety of art-known techniques. For example, soluble polypeptides can be synthesized using standard protein chemistry techniques such as those described in Bodansky, “Principles of Peptide Synthesis,” (Springer Verlag, Berlin (1993)) and Grant (ed.), “Synthetic Peptides: A User's Guide,” (W. H. Freeman and Company, New York (1992)). In addition, automated peptide synthesizers are commercially available (e.g., Advanced ChemTech Model 396; Milligen/Biosearch 9600). Alternatively, soluble polypeptides, fragments or variants thereof may be recombinantly produced using various expression systems as is well known in the art.

Assay Systems for Identifying VEGFR-2 and VEGFR-3 Antagonists

There are numerous approaches to screening for antibody and polypeptide VEGFR-2 and VEGFR-3 antagonists that may be suitably employed in the present methods for inhibiting lymph node lymphangiogenesis and for the treatment of established metastases. For example, antibodies and/or polypeptides that specifically inhibit the binding of a ligand, such as VEGF-C, to a receptor, such as VEGFR-3 or VEGFR-2, can be identified by measuring the inhibition of binding of labeled ligand to a receptor-Fc fusion protein or to a receptor expressing cell. Antibodies and polypeptides identified through this screening approach can then be further tested in animals, as described herein, to assess their ability to inhibit lymph node lymphangiogenesis or established metastases in vivo.

An assay to identify an antibody or soluble peptide that interferes with interaction between a VEGFR-2 or VEGFR-3 and a ligand, such as VEGF-C, can be performed with the component (e.g., cells, purified protein, including fusion proteins and portions having binding activity) which is not to be in competition with a test compound, linked to a solid support. The solid support can be any suitable solid phase or matrix, such as a bead, the wall of a plate or other suitable surface (e.g., a well of a microtiter plate), column pore glass (CPG) or a pin that can be submerged into a solution, such as in a well. Linkage of cells or purified protein to the solid support can be either direct or through one or more linker molecules.

In one embodiment, an isolated or purified protein (e.g., a VEGFR-2 or VEGFR-3 or a ligand, such as VEGF-C) can be immobilized on a suitable affinity matrix by standard techniques, such as chemical cross-linking, or via an antibody raised against the isolated or purified protein, and bound to a solid support. The matrix can be packed in a column or other suitable container and is contacted with one or more compounds (e.g., a mixture) to be tested under conditions suitable for binding of the compound to the protein. For example, a solution containing compounds can be made to flow through the matrix. The matrix can be washed with a suitable wash buffer to remove unbound compounds and non-specifically bound compounds. Compounds which remain bound can be released by a suitable elution buffer. For example, a change in the ionic strength or pH of the elution buffer can lead to a release of compounds. Alternatively, the elution buffer can comprise a release component or components designed to disrupt binding of compounds (e.g., one or more ligands or receptors, as appropriate, or analogs thereof which can disrupt binding or competitively inhibit binding of test compound to the protein).

Fusion proteins comprising all, or a portion of, a protein (e.g., a VEGFR-2 or VEGFR-3 or a ligand, such as VEGF-C) linked to a second moiety not occurring in that protein as found in nature can be prepared for use in another embodiment of the method. Suitable fusion proteins for this purpose include those in which the second moiety comprises an affinity ligand (e.g., an enzyme, antigen, or epitope). The fusion proteins can be produced by inserting the protein (e.g., a VEGFR-2 or VEGFR-3 or a ligand, such as VEGF-C) or a portion thereof into a suitable expression vector which encodes an affinity ligand. The expression vector can be introduced into a suitable host cell for expression. Host cells are disrupted and the cell material, containing fusion protein, can be bound to a suitable affinity matrix by contacting the cell material with an affinity matrix under conditions sufficient for binding of the affinity ligand portion of the fusion protein to the affinity matrix.

In one aspect of this embodiment, a fusion protein can be immobilized on a suitable affinity matrix under conditions sufficient to bind the affinity ligand portion of the fusion protein to the matrix, and is contacted with one or more compounds (e.g., a mixture) to be tested, under conditions suitable for binding of compounds to the receptor or ligand protein portion of the bound fusion protein. Next, the affinity matrix with bound fusion protein can be washed with a suitable wash buffer to remove unbound compounds and non-specifically bound compounds without significantly disrupting binding of specifically bound compounds. Compounds that remain bound can be released by contacting the affinity matrix having fusion protein bound thereto with a suitable elution buffer (a compound elution buffer). In this aspect, compound elution buffer can be formulated to permit retention of the fusion protein by the affinity matrix, but can be formulated to interfere with binding of the compound(s) tested to the receptor or ligand protein portion of the fusion protein. For example, a change in the ionic strength or pH of the elution buffer can lead to release of compounds, or the elution buffer can comprise a release component or components designed to disrupt binding of compounds to the receptor or ligand protein portion of the fusion protein (e.g., one or more ligands or receptors or analogs thereof which can disrupt binding of compounds to the receptor or ligand protein portion of the fusion protein). Immobilization can be performed prior to, simultaneous with, or after contacting the fusion protein with compound, as appropriate. Various permutations of the method are possible, depending upon factors such as the compounds tested, the affinity matrix selected, and elution buffer formulation. For example, after the wash step, fusion protein with compound bound thereto can be eluted from the affinity matrix with a suitable elution buffer (a matrix elution buffer). Where the fusion protein comprises a cleavable linker, such as a thrombin cleavage site, cleavage from the affinity ligand can release a portion of the fusion with compound bound thereto. Bound compound can then be released from the fusion protein or its cleavage product by an appropriate method, such as extraction.

### Methods for Treatment of Metastatic Disease and Inhibition of Lymphangiogenesis

In certain embodiments, the present invention provides methods for inhibiting lymph node, lung, liver, kidneys, skin, peritoneum, or other distant organ lymphangiogenesis and methods for inhibiting established tumor metastases (e.g., for metastatic tumors derived from tumors such as, e.g., breast, colorectal, prostate, pancreas, head-and-neck, renal, lung, skin, etc.). These methods involve administering to a subject in need thereof (e.g., a human or veterinary animal) a therapeutically effective amount of one or more VEGFR-3 antagonist(s) and optionally one or more VEGFR-2 antagonist(s) (administered in one or more compositions, the latter administered simultaneously or sequentially).

These methods are particularly aimed at the therapeutic treatment of established metastatic disease in animals, in particular humans. Thus, the methods disclosed herein are most suitably employed for the prevention of cancer recurrence and inhibition of metastases after eradication and/or removal of a primary tumor, such as, e.g., by surgery, chemotherapy, radiation therapy, phototherapy, and/or immunotherapy. The methods disclosed herein can also be employed for inhibition of metastases in patients in which primary tumor cannot be removed.

In certain embodiments of such methods, VEGFR-3 or VEGFR-2 antagonist(s) can be administered together with a radiation treatment and/or with one or more additional compound(s) useful for inhibiting lymphangiogenesis and/or metastasis.

It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Non-limiting examples of chemotherapeutic compounds which can be used in combination treatments of the present invention include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that can be used in combination with a VEGFR-3 or VEGFR-2 antagonist include: (1) inhibitors of release of “angiogenic molecules,” such as bFGF (basic fibroblast growth factor); (2) neutralizers of angiogenic molecules, such as an anti-ObFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Bioch. Biophys. Acta. 1032:89-118 (1990), Moses et al., Science 248:1408-1410 (1990), Ingber et al., Lab. Invest. 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of the polypeptide therapeutic agents of the disclosure may be continued while the other therapy is being administered and/or thereafter. Administration of the therapeutic agents can be made in a single dose, or in multiple doses. In some instances, administration of the therapeutic agents can be commenced at least several days prior to the conventional therapy, while in other instances, administration can begin either immediately before or at the time of the administration of the conventional therapy.

### Methods for Administering Compositions Comprising VEGFR-3 and VEGFR-2 Antagonists

In certain embodiments, the VEGFR-3 or VEGFR-2 antagonist(s) are formulated in pharmaceutical compositions with a pharmaceutically acceptable carrier or excipient. The compounds can be formulated for administration in any convenient way for use in human or veterinary medicine. Wetting agents, emulsifiers and lubricants, such as sodium lauryl sulfate and magnesium stearate, as well as coloring agents, release agents, coating agents, preservatives and antioxidants can also be present in the compositions.

Formulations of VEGFR-3 or VEGFR-2 antagonist(s) useful in the methods of the invention include those suitable for oral/nasal, topical, and/or parenteral administration. The formulations may conveniently be presented in unit dosage form and may be prepared by any methods well known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

In certain embodiments, methods of preparing these formulations or compositions include combining another type of anti-tumor or anti-angiogenesis therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, powders, granules, or as a solution or a suspension in an aqueous or non-aqueous liquid, or as an oil-in-water or water-in-oil liquid emulsion, and the like, each containing a predetermined amount of one or more active ingredients.

In solid dosage forms for oral administration (capsules, tablets, pills, dragees, powders, granules, and the like), one or more active ingredients can be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Suspensions, in addition to one or more active ingredients, can contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Compositions of the invention can be also administered topically, either to skin or to mucosal membranes. This offers the greatest opportunity for direct delivery with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject therapeutic agents may be mixed under sterile conditions with a pharmaceutically acceptable carrier, and with any preservatives, buffers, or propellants which may be required. The ointments, pastes, creams and gels may contain, in addition to a subject polypeptide agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to one or more active ingredients, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more active ingredients in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more active ingredients in biodegradable polymers such as polylactide-polyglycolide. Depending on the ratio of active ingredient to polymer, and the nature of the particular polymer employed, the rate of antagonist release can be controlled. Examples of other biodegradable polymers include poly(orthoesters) and poly(anhydrides). Depot injectable formulations are also prepared by entrapping the antagonists in liposomes or microemulsions which are compatible with body tissue.

Formulations for intravaginal or rectal administration may be presented as a suppository, which may be prepared by mixing one or more active ingredients with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In other embodiments, one or more VEGFR-2 antagonist or one or more VEGFR-3 antagonist can be expressed within cells from eukaryotic promoters. For example, one or more VEGFR-2 antagonist or one or more VEGFR-3 antagonist can be expressed in eukaryotic cells from an appropriate vector. The vectors are preferably DNA plasmids or viral vectors. Viral vectors can be constructed based on, but not limited to, adeno-associated virus, retrovirus, adenovirus, or alphavirus. Preferably, the vectors stably introduced in and persist in target cells. Alternatively, viral vectors can be used that provide for transient expression. Such vectors can be repeatedly administered as necessary. Delivery of vectors encoding the antagonists can be systemic, such as by intravenous or intramuscular administration, by administration to target cells ex-planted from the patient followed by reintroduction into the patient, or by any other means that would allow for introduction into the desired target cell (for a review see Couture et al., TIG 12:510 (1996).

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Inhibition of Lymph Node Lymphangiogenesis by Combined Inhibition of VEGFR-2 and VEGFR-3 Signaling for Inhibition of Regional and Distant Metastases

It has been reported that systemic treatment with VEGFR-3 antagonistic antibodies (mF4-31C1, ImClone Systems a subsidiary of Eli Lilly and Company, Indianapolis, Ind.) suppressed tumor lymphangiogenesis and inhibited lymph node metastasis of MDA-MB-435 cells expressing high levels of VEGF-C (MDA/VEGF-C). Furthermore, a combination therapy with anti-VEGFR-2 and anti-VEGFR-3 antibodies has been shown to be more potent in decreasing lymph node metastases than treatment with either antibody alone. Roberts et al., Cancer Res. 66:2650-7 (2006). The effects of a combination treatment were studied in an intervention regimen, in which the treatment commenced when tumors and metastases were established, four weeks after the orthotopic tumor cell inoculation into the mammary fat pads. Combined treatment with the antagonistic antibodies to VEGFR-2 and VEGFR-3 also significantly decreased the amount of lung metastases, suggesting that a combination therapy with anti-angiogenic and anti-lymphangiogenic agents may be a particularly promising approach for control of tumor growth and metastases.

To understand the mechanism by which combined treatment inhibits metastasis, its effects on the primary tumor were investigated. Interestingly, combined treatment did not result in greater inhibition of primary tumor growth than treatment with the anti-VEGFR-2 antibody alone (blocking VEGFR-3 had no effect on primary tumor growth). Analysis of tumor vasculature showed that double-treatment was also not more potent in inhibiting tumor lymphangiogenesis or angiogenesis, as compared to single antibody treatments. These data (summarized in Table 2) demonstrate that the effects of combined treatment on metastases can not be explained by changes of the tumor vasculature or growth of the primary tumor.

TABLE 2
Effects of Treatments with mF4-31C1 (anti-VEGFR-3)
and DC101 (anti-VEGFR-2) Antibodies on Tumor
Growth, Vascularization, and Metastasis

| | MDA-MB-435/VEGF-C | | anti-VEGFR-3 + |
|---|---|---|---|
| | anti-VEGFR-3 | anti-VEGFR-2 | VEGFR-2 |
| tumor growth | − | +++ | +++ |
| tumor angiogenesis | −/+ | +++ | +++ |
| tumor | +++ | ++ | +++ |
| lymphangiogenesis | | | |
| LN metastasis, | +++ | + | N/A |
| prevention | | | |
| LN metastasis, | + | − | +++ |
| intervention | | | |
| Lung metastasis, | +++ | ++ | N/A |
| prevention | | | |
| Lung metastasis, | + | − | ++ |
| intervention | | | |

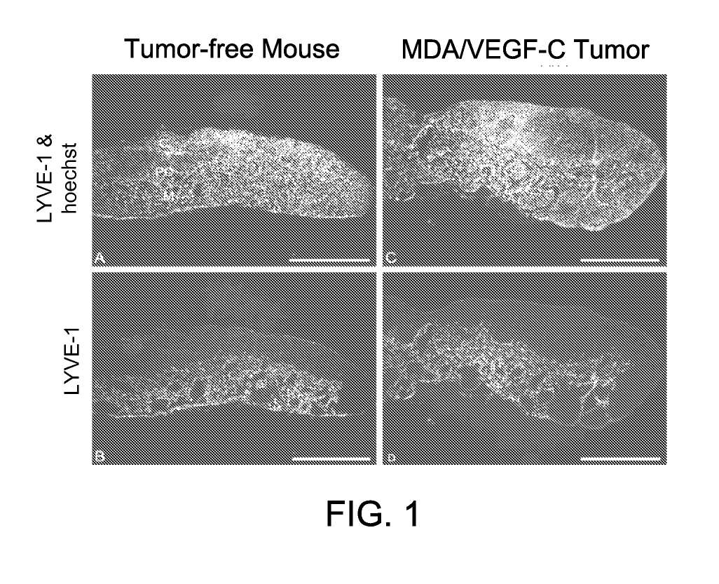

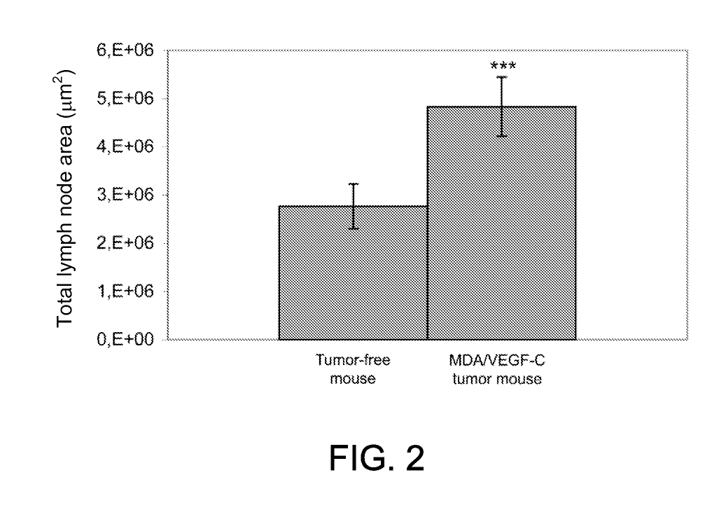

Based on these findings, it was hypothesized that events downstream from the primary tumor, i.e. in the lymph nodes, may be important for the observed inhibition of metastases with the combined treatment. To test this hypothesis, the pattern of lymphatic and blood vasculature in tumor draining lymph nodes of control and treated mice was examined by immunofluorescent staining using LYVE-1 and CD34 antibodies, respectively. The results showed that MDA-MB-435/VEGF-C tumors induced prominent lymphangiogenesis in tumor draining axillary lymph nodes. LYVE-1⁺ lymphatic vessels were restricted to medullary zone and subcapsular sinuses, whereas no LYVE-1⁺ structures were seen in the lymph node cortex (FIG. 1). In addition, tumor draining lymph nodes were greatly increased in size when compared to control lymph nodes of normal mice (FIG. 2).

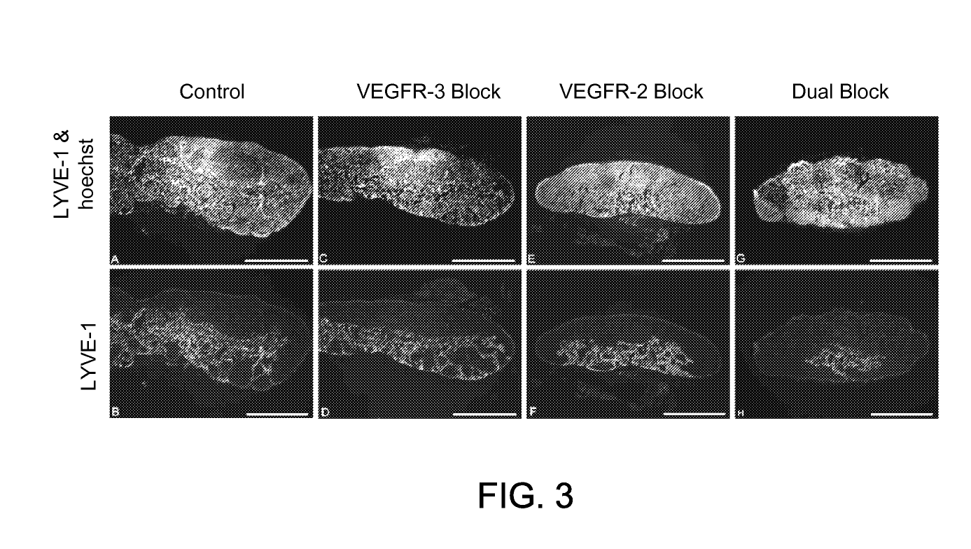

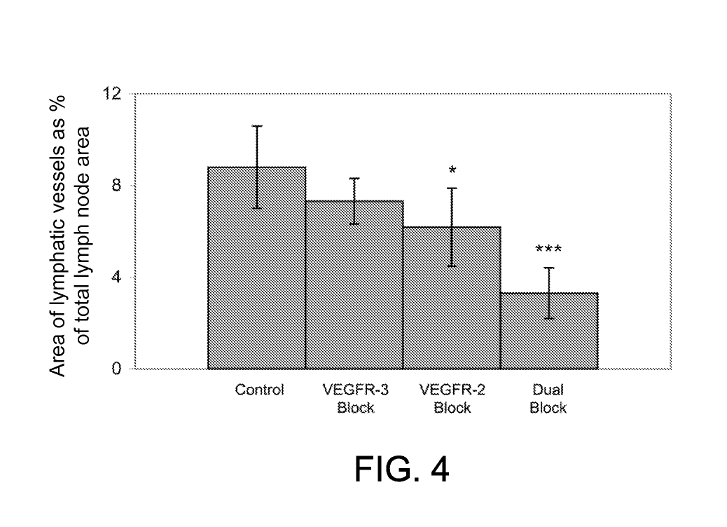

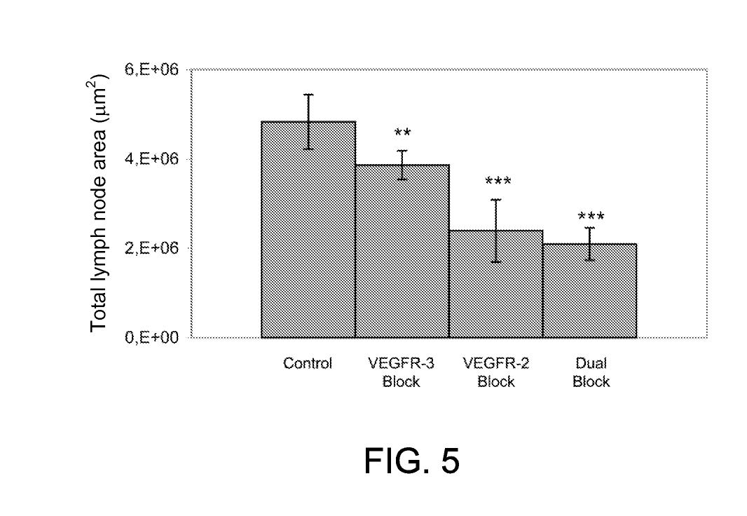

Blocking VEGFR-3 reduced the lymphatic vessel area and lymph node size in the tumor-draining lymph nodes to a moderate extent. Blocking VEGFR-2 showed moderate inhibition of lymphangiogenesis and a more prominent inhibition of lymph node size. Quite surprisingly, however, the combined blocking of VEGFR-3 and VEGFR-2 drastically inhibited lymph node lymphangiogenesis (FIGS. 3 and 4) and dramatically reduced lymph node size (FIGS. 3 and 5).

Taken together, analysis of tumor draining lymph nodes revealed that combined blocking of VEGFR-3 and VEGFR-2 was most effective in inhibiting lymph node lymphangiogenesis and lymph node size, as compared to single antibody treatments. These data demonstrated the importance of concurrent VEGFR-2 and VEGFR-3 signaling for lymph node lymphangiogenesis and strongly indicated an important role of lymph node lymphangiogenesis for both lymph node and distant metastases.

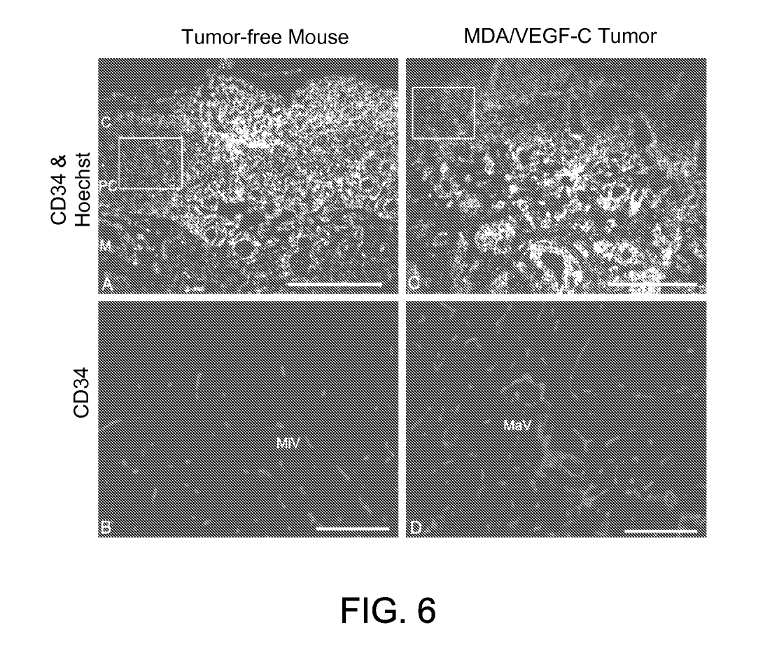

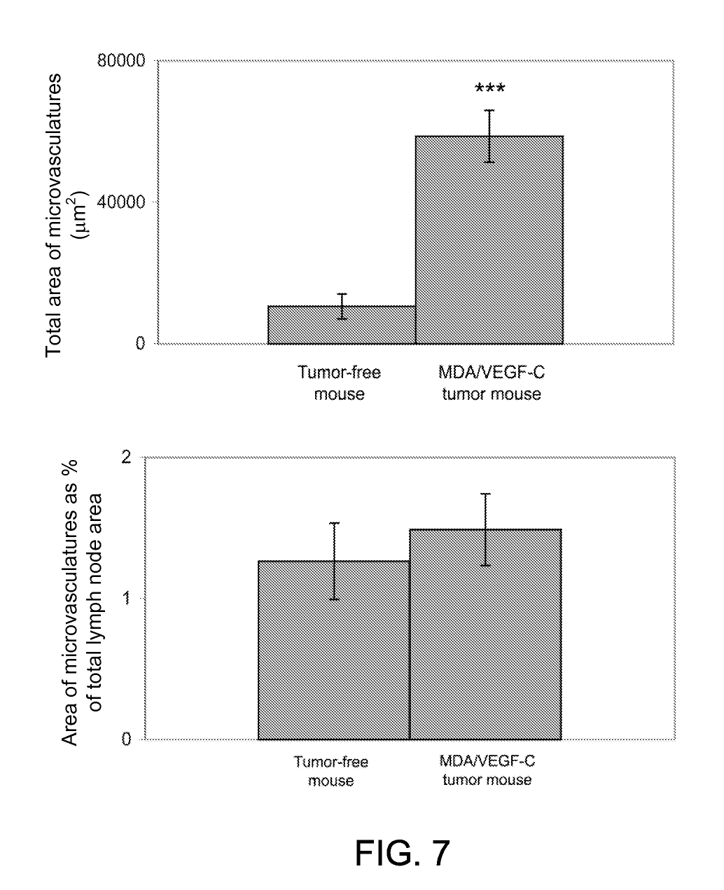

The effects of antagonistic antibodies to VEGFR-2 and VEGFR-3 on tumor-induced lymph node angiogenesis were examined. Comparison of the CD34⁺ vessel pattern in normal and tumor draining lymph nodes showed an increase in the density of blood microvasculature within the lymph node cortex, whereas the density of blood macrovasculature was not altered. Tumors induced angiogenesis in tumor-draining lymph nodes by 80%. Interestingly, the increase in total number of blood vessels was directly correlated to the increase of lymph node size associated with the presence of the tumor indicating that the blood vessel density per lymph node area remained unchanged (FIGS. 6 and 7).

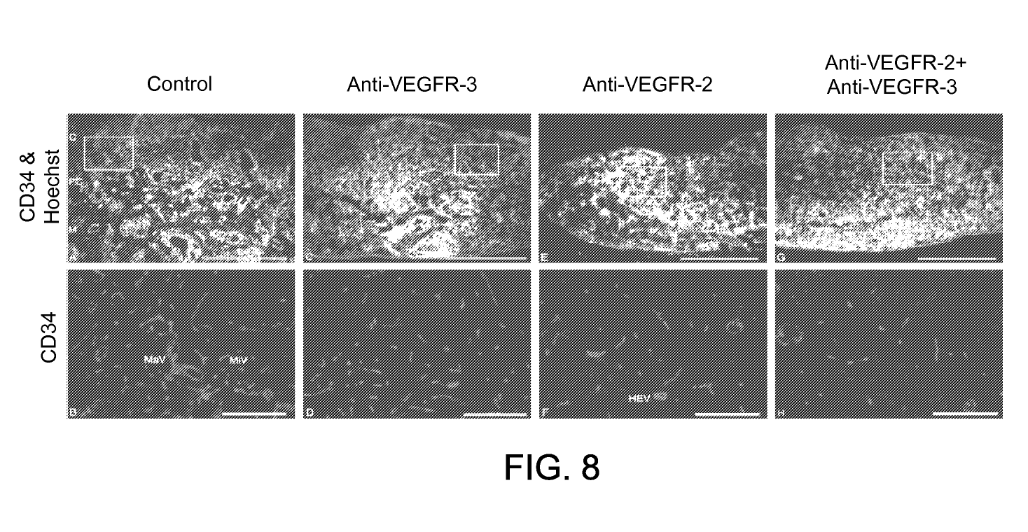

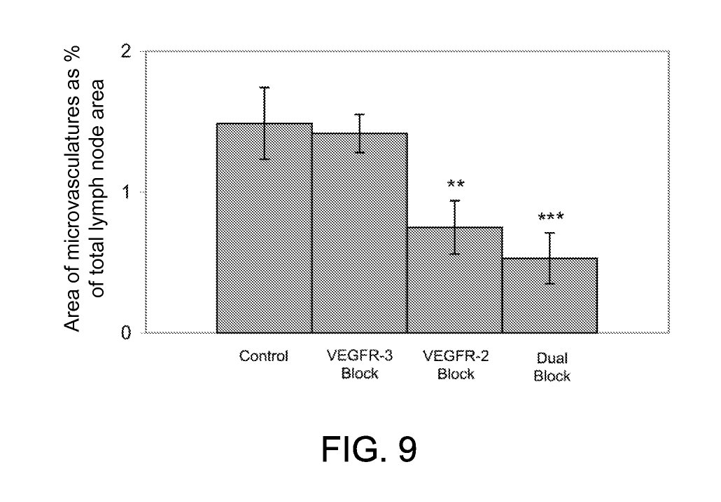

Blocking VEGFR-3 alone did not result in reduction of the lymph node blood vessel density. Blocking VEGFR-2, however, drastically reduced blood vessel density (50%). Combined blocking of VEGFR-2 and VEGFR-3 reduced blood vessel numbers slightly more (64%) (FIGS. 8 and 9, respectively). Thus, the combination treatment, with antagonistic antibodies to both VEGFR-2 and VEGFR-3 is most effective for the inhibition of lymph node lymphangiogenesis and lymph node size. These data are summarized in Table 3.

TABLE 3
Effects of Treatments with mF4-31C1 (anti-
VEGFR-3) and DC101 (anti-VEGFR-2) Antibodies
on Lymph Node Size and Vascularization

| | mF4-31C1 | DC101 | mF4-31C1 + DC101 |
|---|---|---|---|
| LN angiogenesis | − | ++ | ++ |
| LN lymphangiogenesis | + | + | +++ |
| LN size | + | ++ | +++ |

In summary, analysis of tumor-draining lymph nodes revealed that combined blocking of VEGFR-3 and VEGFR-2 was most effective in inhibiting lymph node lymphangiogenesis, as compared to single antibody treatments. Furthermore, these results demonstrated that concurrent VEGFR-2 and VEGFR-3 signaling is important for lymphangiogenesis in the lymph nodes, but not at the primary tumor site. While VEGFR-3 was necessary for lymphangiogenesis, blocking VEGFR-3 was not sufficient to completely inhibit lymph node lymphangiogenesis, indicating the differences in the mechanism of lymphangiogenesis at different anatomical sites. Finally, these findings also revealed that tumor-induced expansion of the lymphatic vasculature in the lymph nodes, mediated by concurrent VEGFR-2 and VEGFR-3 signaling, was important for tumor metastasis to lymph nodes and to distant sites.

### Example 2

### Inhibition of VEGFR-3 Signaling for Treatment of Established Metastatic Disease

This Example discloses the inhibition of VEGFR-3 signaling or combined VEGFR-3 and VEGFR-2 signaling for treatment of established metastatic disease. VEGF-C overexpressing tumors increased lung metastases and induced an endovascular pulmonary metastatic phenotype in the peribronchovascular region.

VEGF-C expression by tumor cells potently increases metastatic burden in the lungs. Skobe et al., Nature Med. 7:192-198 (2001). To further understand the mechanism by which VEGF-C and its receptors facilitate formation of distant metastases, the phenotype of lung metastases formed by MDA-MB-435 and MDA-MB-435/VEGF-C cells was examined. These cells were injected orthotopically into the 2nd mammary fat pads of nude mice and tumors and metastases were allowed to develop for 12 weeks. Tumor size reached an average volume of ˜1 cm³ after the 12 week period. At the end of the experiment (12 weeks), 8 out of 8 mice (100%) bearing MDA-MB-435 cells or MDA-MB-435/VEGF-C cells had a positive signal in the lungs.

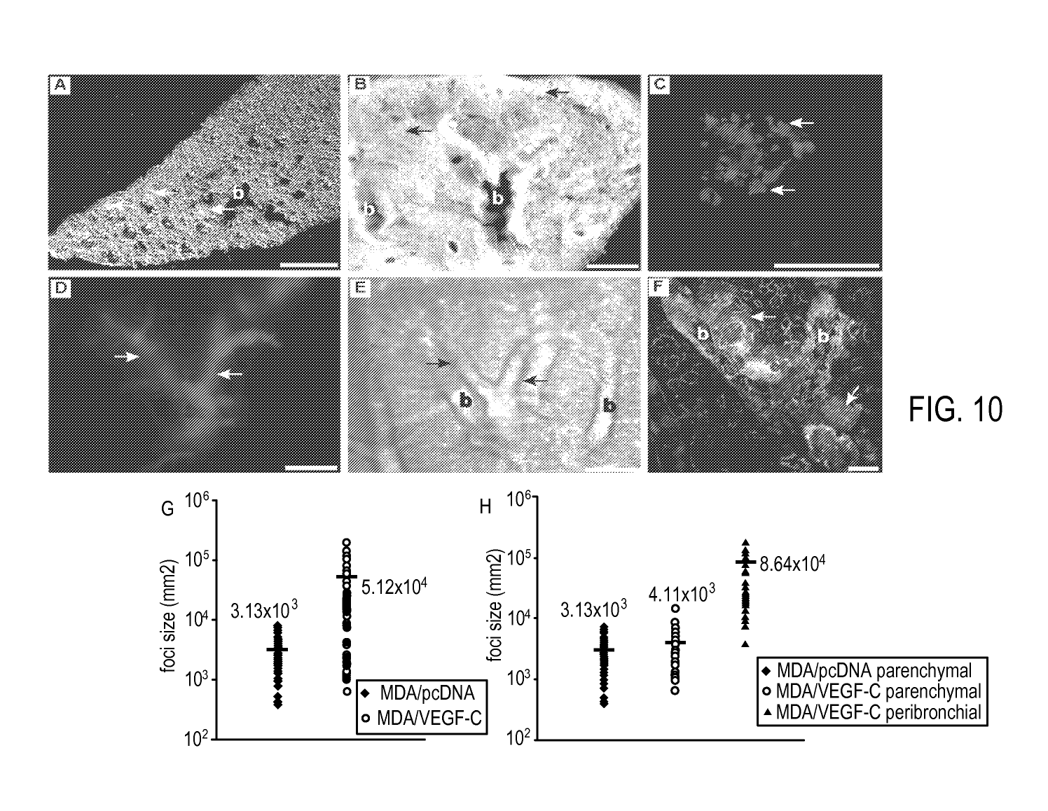

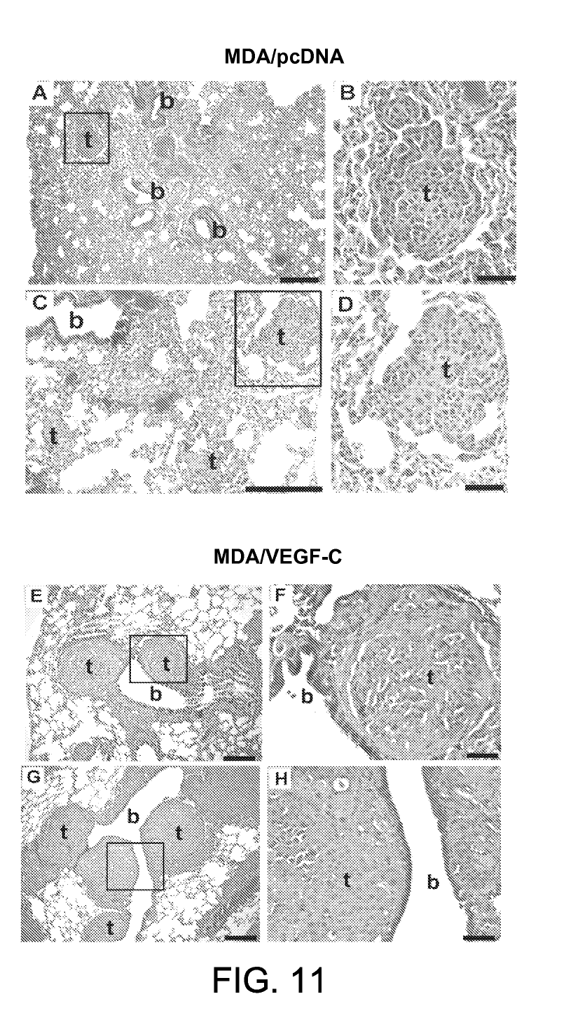

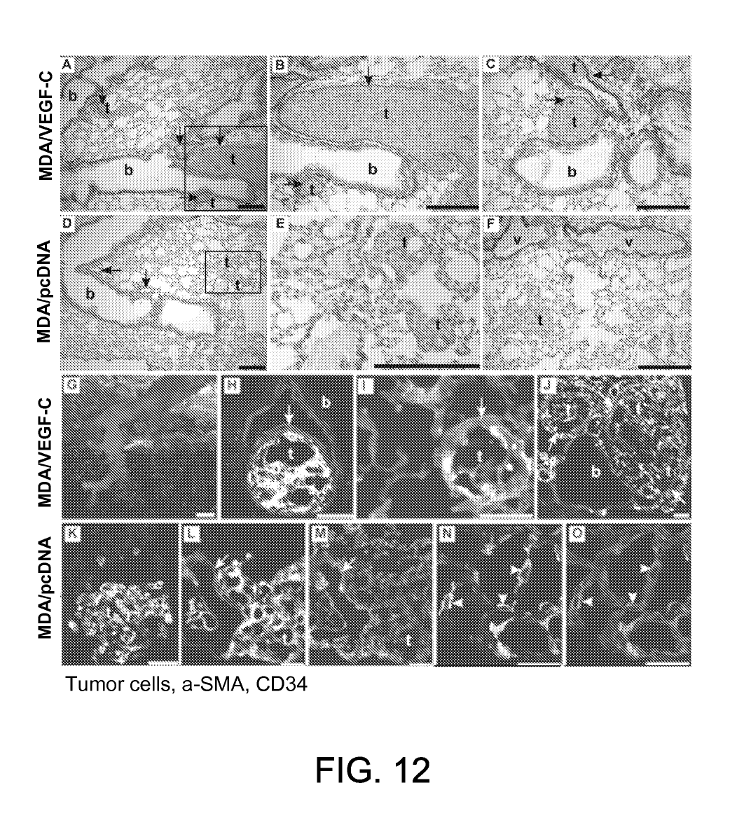

Interestingly, histopathological analysis of metastases revealed a distinct pattern of pulmonary metastases by tumor cells expressing high levels of VEGF-C. In addition to an increase in overall lung metastases, MDA-MB-435/VEGF-C cells showed a unique distribution in the lung as compared to the non-VEGF-C expressing cells (FIGS. 10 and 11). Metastases from MDA-MB-435/VEGF-C cells presented as large lesions predominately associated with the bronchi and large pulmonary vessels. In contrast, metastases of MDA-MB-435 control cells presented as small pulmonary nodules that localized in the lung parenchyma and were not associated with the bronchi. Smooth muscle α-actin staining of the large pulmonary vessels and airways further showed that metastases from MDA-MB-435 cells had no affiliation with the large pulmonary vasculature and were often distant from large vessels, whereas VEGF-C overexpressing lesions were often seen in the form of intravascular emboli, localizing in pulmonary arteries (FIG. 12).

These results demonstrate that increased VEGF-C production by metastatic cells resulted in an increase in lung metastasis and drives a phenotype in which tumor cells are frequently seen as endovascular nodules in the peribronchovascular region.

### Example 3

### VEGF-C Induces Dilation of Pulmonary Lymphatics and Metastatic Lymphangiogenesis

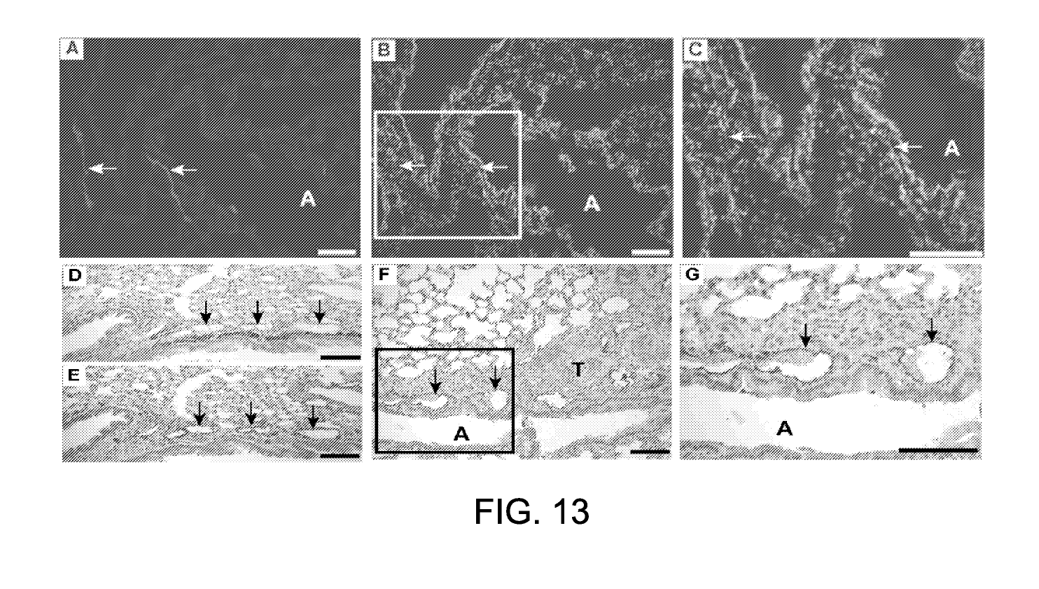

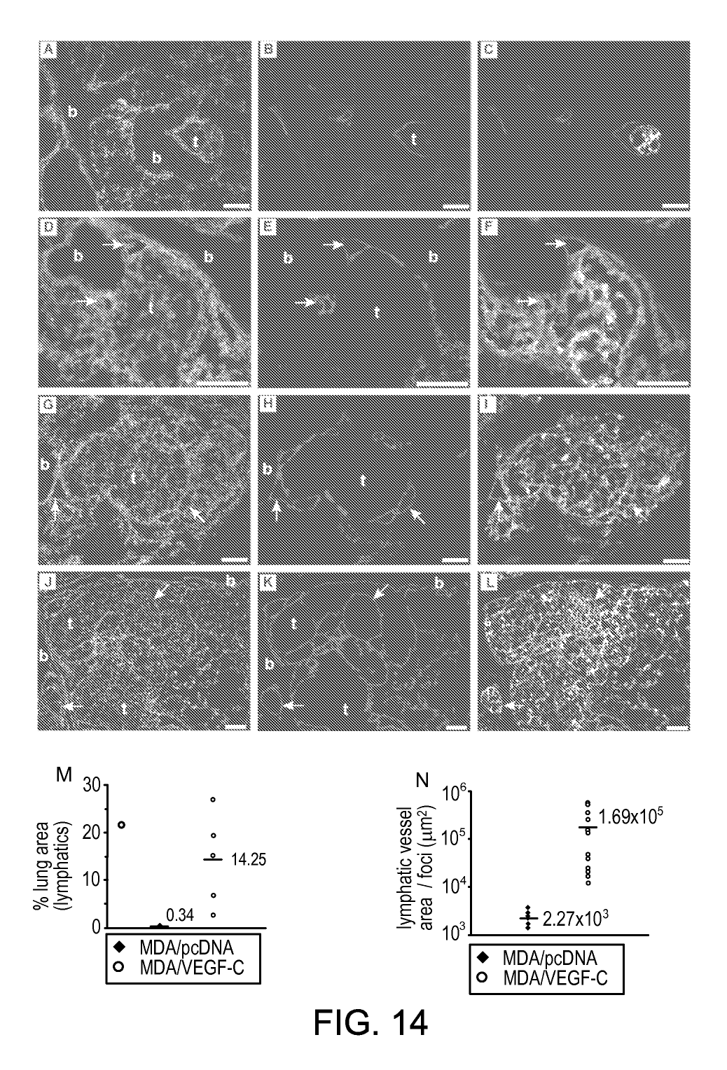

Lymphatic vessels in lungs infiltrated with MDA/pcDNA tumor cells were detected in their normal anatomical location, i.e. surrounding bronchi, large pulmonary vessels and in the pleura, and they were not altered in their appearance as compared to normal lungs not involved with cancer. There was no lymphangiogenesis associated with MDA/pcDNA nodules, and only seldom were lymphatics seen in the vicinity of these nodules (number of metastatic foci with lymphatics present within 200 μm: MDA/pcDNA 6/48; 13% vs. MDA/VEGF-C 37/39; 95%). In contrast, VEGF-C-overexpressing metastatic lesions were characterized by pronounced lymphangiogenesis and lymphatic vessels were greatly distended throughout the lungs with MDA/VEGF-C metastases (FIGS. 13 and 14). Lymphatic vessel area associated with MDA/VEGF-C metastatic foci was on average 75-fold greater than lymphatic vessel area associated with MDA/pcDNA foci which had lymphatics in the proximity. Expansion of lymphatic network paralleled an increase in size of MDA/VEGF-C metastases. In summary, this data shows that VEGF-C can drastically alter architecture of pulmonary lymphatic vasculature by inducing lymphangiogenesis and lymphatic vessel enlargement.

### Example 4

### VEGF-C Promotes Intralymphatic Spread of Metastases in the Lung

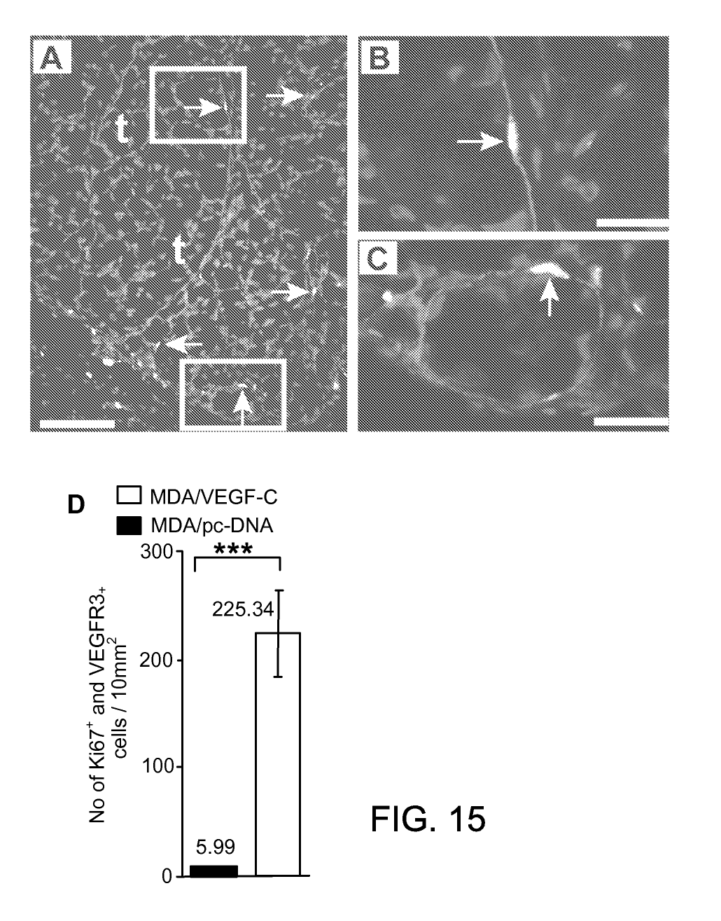

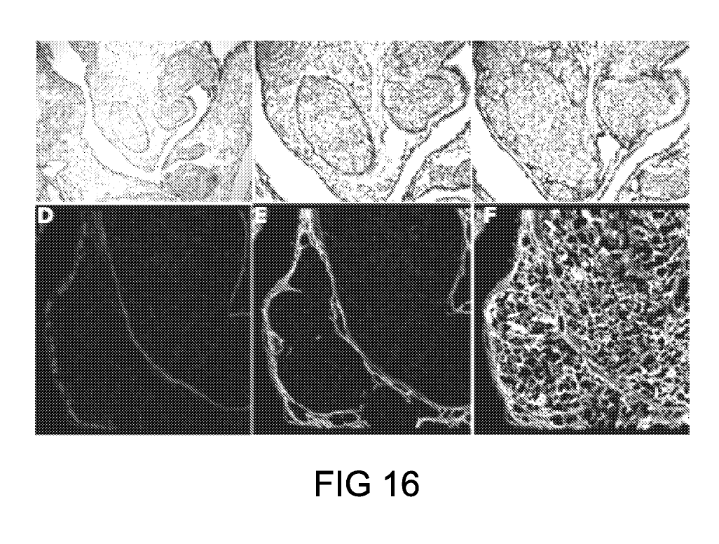

Because majority of the VEGF-C overexpressing metastases were found adjacent to bronchi, the relationship between MDA/VEGF-C metastases and the deep lymphatic plexus associated with the bronchial tree was investigated. Lymphatic vessels were detected by using a combination of anti-LYVE-1, anti-podoplanin, and anti-VEGFR-3 antibodies. Strikingly, bulk of MDA/VEGF-C metastases observed in the peribronchial area was seen inside of the greatly distended lymphatic vessels (31/55 metastatic foci showed lymphatic vessel involvement; 56%) (FIGS. 15 and 16). Lymphatic vessels in the proximity of pulmonary veins were also massively infiltrated with tumor cells. Furthermore, MDA/VEGF-C metastases were frequently detected in the pleura (MDA/VEGF-C in 5/7 mice; MDA/pcDNA in 1/6 mice), which is another area of lung tissue rich in lymphatics. In sharp contrast, MDA/pcDNA metastases were rarely seen intravascular or even in the vicinity of the lymphatic vasculature. These data indicate that VEGF-C facilitates intralymphatic spread of metastases in the lung.

### Example 5

### VEGF-C Increases Thoracic Lymph Node Metastases

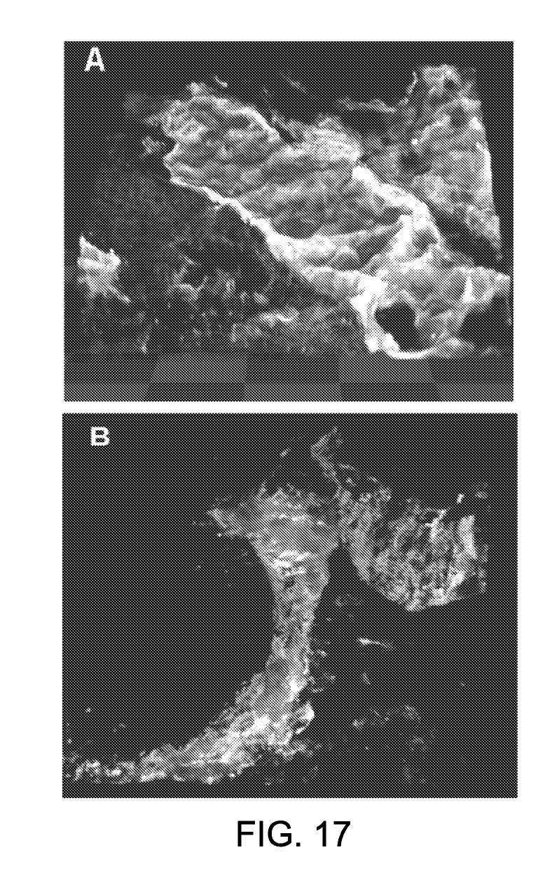

To investigate whether VEGF-C promotes secondary metastatic dissemination, within the lymphatic network in the lung, lung-draining lymph nodes were analyzed for the presence of metastases. At necropsy, mediastinal and hilar lymph nodes from mice bearing VEGF-C overexpressing and control MDA-MB-435 tumors were evaluated using stereomicroscopy to detect the GFP fluorescence. The incidence of lymph nodes positive for metastases from VEGF-C overexpressing tumor bearing mice was significantly higher (13/20, 65%) than the incidence in mice bearing control tumors (3/20, 15%). To confirm the presence of tumor cells and to further analyze the specific area of the metastases within the mediastinal or hilar lymph nodes, the histopathology of lymph nodes from mice bearing the VEGF-C over-expressing tumors was studied (FIG. 17). Using podoplanin as a lymphatic vessel marker, metastases were predominately observed in the subcapsular sinus region. These data demonstrate that VEGF-C promotes lymphatic spread of metastases within the lung, promoting secondary tumor spread to the thoracic lymph nodes.

### Example 6

### Inhibition of VEGFR-3 Reverses the Lymphangitic Carcinomatosis Metastatic Phenotype

The finding that VEGF-C induces aggressive Lymphangitic Carcinomatosis phenotype in a mouse breast cancer model suggested that VEGF-C and its receptor VEGFR-3 and/or VEGFR-2 could be targets for treatment of this disease. Because in most cancer patients with cancer primary tumor is removed (unless it is unresectable), an experiment was designed in which VEGFR-3 signaling was inhibited by use of antagonistic antibodies after the removal of the primary tumor. VEGF-C overexpressing MDA-MB-435 cells were orthotopically injected into the 2nd mammary fat pad of nu/nu mice and tumor and lung metastases were allowed to develop for 8 weeks. The extent of metastases was monitored and quantified by in vivo bioluminescent imaging. At the 8^{th} week, the primary tumor was surgically removed and function-blocking antibodies to VEGFR-3 (mF4-31C1, ImClone Systems) were administered at 800 μg/mouse every second day and metastases were analyzed after six weeks of treatment.

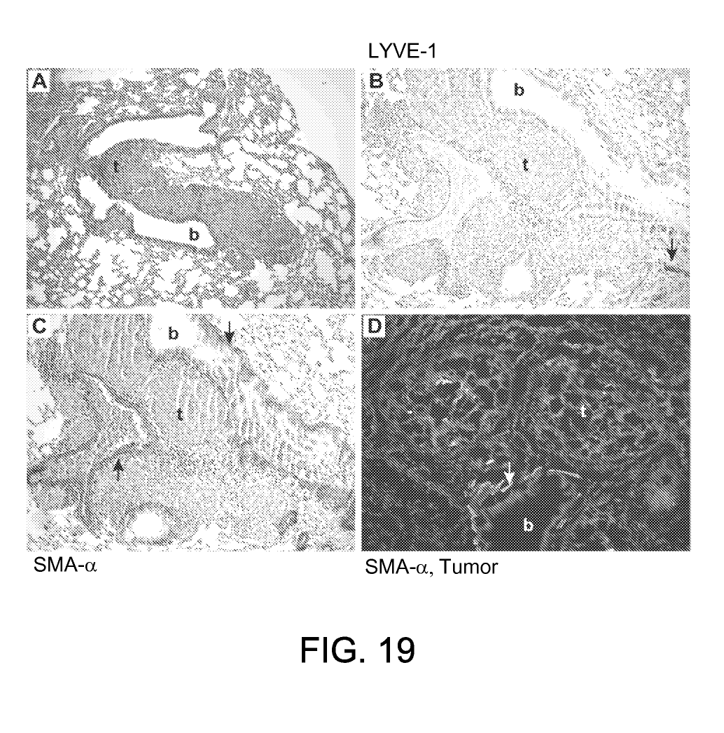

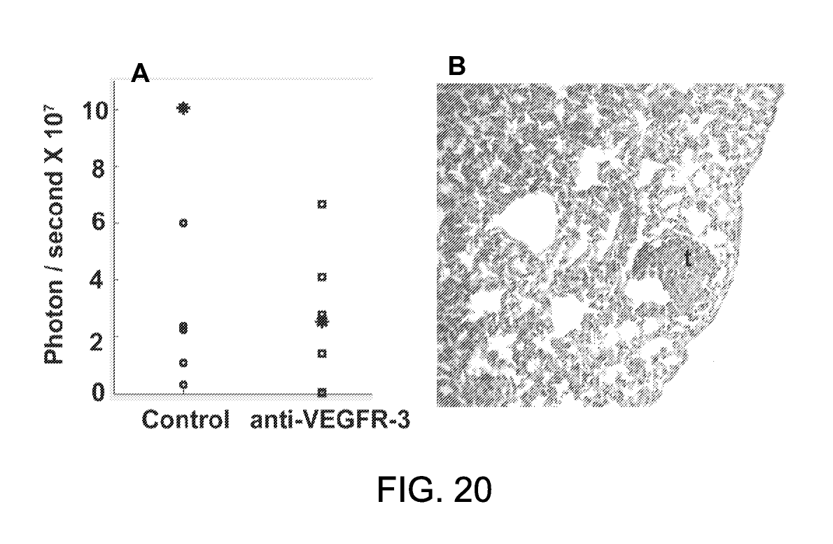

A stark contrast between the metastatic patterns between the control and the mF4-31C1 treated groups was observed, as assessed by histopathological analysis of lung sections. As shown in FIGS. 19 and 20, control samples (MDA/VEGF-C cells) exhibited many intravascular metastases observed in the pulmonary arteries associated with the bronchial tree, and in the lymphatic vessels of the peribronchovascular region, around the pulmonary veins, and in the pleura. Conversely, in lung sections from the mice treated with anti-VEGFR-3 antibodies, metastases were observed primarily in the lung parenchyma and capillaries of the lungs, and were less frequently associated with lymphatics, pulmonary arteries, or the bronchial tree. Metastases associated with the airways were not found in the lymphatic vessels or in the pulmonary arteries, and there was no lymphangiogenesis. Compare with FIG. 16, which shows typical phenotype of VEGF-C expressing metastases which grow in the lymphatic vessels and present as pulmonary artery tumor emboli. Collectively, these data demonstrate that VEGF-C/VEGFR-3 signaling has a significant role in driving breast adenocarcinoma metastases towards the clinical manifestation of Lymphangitic Carcinomatosis and that inhibition of VEGFR-3 can reverse this aggressive phenotype.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A method for inhibiting an established tumor metastasis in a subject comprising administering to said subject a therapeutically effective amount of one or more VEGFR-3 antagonist(s) and optionally one or more VEGFR-2 antagonist(s).

2. The method of claim 1, wherein said antagonist(s) is administered after the eradication or removal of a primary tumor.

3. The method of claim 1, wherein said metastasis is in a distant organ.

4. The method of claim 3, wherein the distant organ is selected from the group consisting of lung, liver, kidney, peritoneum, and skin.

5. The method of claim 1, wherein said metastasis is in a lymph node.

6. The method of claim 1, wherein said metastasis is diagnosed as Lymphangitic Carcinomatosis.

7. A method for inhibiting lymphangiogenesis in a subject with a metastatic disease comprising administering to said subject a therapeutically effective amount of one or more VEGFR-3 antagonist(s) and optionally one or more VEGFR-2 antagonist(s).

8. The method of claim 1 or claim 7, wherein said antagonist is an antibody or an antigen-binding portion thereof.

9. The method of claim 8, wherein said antibody is capable of binding to an extracellular domain of VEGFR-2 or VEGFR-3.

10. The method of claim 8, wherein said antibody is capable of blocking the interaction of VEGF-C with VEGFR-2 or VEGFR-3.

11. The method of claim 8, wherein said antibody binds to its target with an affinity of at least about 1×10⁻⁶ M.

12. The method of claim 8, wherein said antibody binds to its target with an affinity of at least about 1×10⁻⁸ M.

13. The method of claim 8, wherein the antigen-binding portion of said antibody is selected from the group consisting of an F(ab′)₂, a Fab, an Fv, an scFv, and a single domain antibody.

14. The method of claim 8, wherein said antibody is selected from the group consisting of a chimeric antibody, a primatized antibody, and a humanized antibody.

15. The method of claim 14, wherein said humanized antibody contains one or more CDR(s) from monoclonal antibody DC101 or monoclonal antibody mF4-31C1.

16. The method of claim 1, wherein said antagonist is a soluble polypeptide antagonist.

17. The method of claim 16, wherein said soluble polypeptide antagonist comprises an extracellular domain of a VEGFR-2 protein or an extracellular domain of a VEGFR-3 protein.

18. The method of claim 16, wherein said soluble polypeptide antagonist comprises an amino acid sequence that is at least 90% identical to the extracellular domain of a VEGFR-2 protein.

19. The method of claim 16, wherein said soluble polypeptide antagonist comprises an amino acid sequence that is at least 90% identical to the extracellular domain of a VEGFR-3 protein.

20. The method of claim 16, wherein said soluble polypeptide antagonist further comprises a post-translational modification selected from the group consisting of acetylation, carboxylation, glycosylation, phosphorylation, lipidation, and acylation.

21. The method of claim 16, wherein said soluble polypeptide antagonist further comprises a non-amino acid element selected from the group consisting of a polyethylene glycol, a lipid, a poly- or mono-saccharide, and a phosphate.

22. The method of claim 16, wherein said soluble polypeptide antagonist further comprises a fusion domain selected from the group consisting of polyhistidine, Glu-Glu, glutathione S transferase (GST), thioredoxin, protein A, protein G, an immunoglobulin heavy chain constant region (Fc), a maltose binding protein (MBP), green fluorescent protein (GFP), and an epitope tag.

23. The method of claim 22, wherein said fusion domain further comprises a protease cleavage site selected from the group consisting of Factor Xa and Thrombin.

24. The method of claim 1 or claim 7, wherein said antagonist is administered in combination with a radiation treatment or with one or more additional compound(s) useful for inhibiting lymphangiogenesis or metastasis.

25. The method of claim 24, wherein said additional compound is a chemotherapeutic.

26. The method of claim 24, wherein said additional compound is an anti-angiogenic compound.
